(19) United States
(12) Patent Application Publication
(10) Pub. No.: US 2024/0366261 A1
(43) Pub. Date: Nov. 7, 2024

(54) FLEXIBLE TUBULAR ELONGATE MEMBER FOR MEDICAL USE, AND ASSOCIATED SYSTEMS AND METHODS

(71) Applicant: Boston Scientific Scimed, Inc., Maple Grove, MN (US)

(72) Inventors: Vladimir Mitelberg, Austin, TX (US); John Mims, Austin, TX (US); Ryan Gilkey, Kyle, TX (US)

(73) Assignee: Boston Scientific Scimed, Inc., Maple Grove, MN (US)

(21) Appl. No.: 18/655,133

(22) Filed: May 3, 2024

Related U.S. Application Data
(60) Provisional application No. 63/541,160, filed on Sep. 28, 2023.
(60) Provisional application No. 63/464,007, filed on May 4, 2023.

(51) Int. Cl. A61B 17/34; A61B 17/00; A61B 17/04; A61M 25/00

(52) U.S. Cl. CPC A61B 17/3421; A61B 17/0469; A61B 2017/0034; A61M 25/0045; A61M 25/0054; A61M 2025/0059

(57) ABSTRACT

A kink-resistant and/or deformation-resistant flexible tubular elongate member capable of being retroflexed greater than 90°, such as greater than 180°, without one or more lumens therethrough kinking, deforming, distorting, ovalizing, elongating, or otherwise changing shape and/or otherwise being affected functionally.

### CROSS-REFERENCE TO RELATED APPLICATIONS

This application claims the benefit of priority under 35 U.S.C. § 119 to U.S. Provisional Application No. 63/541,160, filed Sep. 28, 2023, and U.S. Provisional Application No. 63/464,007, filed May 4, 2023, the entire disclosures of which are hereby incorporated by reference herein for all purposes. Any and all priority claims identified in the Application Data Sheet, or any correction thereto, are hereby incorporated by reference under 37 C.F.R. § 1.57.

### FIELD

The present disclosure relates to devices, systems, and methods for use during retroflexion of a medical device. More particularly, the present disclosure relates to devices and/or systems which are formed so as not to be distorted during or by retroflexion of the medical device. Even more particularly, the present disclosure relates to flexible tubular elongate members which maintain patency of a lumen extending therethrough while being retroflexed. The flexible tubular elongate member may be configured to be mounted with respect to a medical device which is retroflexed.

### BACKGROUND

Various medical devices are designed to be inserted into the body of a patient and articulated, flexed, retroflexed, etc., within the patient's body. For instance, one or more devices, instruments, tools, etc., may be advanced to a target site within a patient's body through one or more working channels of a medical scope (e.g., endoscope). Such devices may also be known as through-the-scope (“TTS”) devices. Some TTS devices may include one or more flexible tubular elongate members through which a further component, device, instrument, tool, etc., may be extended through the medical scope. Additionally or alternatively, over-the-scope (“OTS”) endoscopic accessories may be extended along (and optionally attached to) the exterior surface of a medical scope. Various OTS devices may perform various functions that work in cooperation with the associated medical scope and one or more TTS devices. Some OTS accessory devices may include one or more flexible tubular elongate members defining a lumen extending longitudinally therethrough. The lumen may function as an auxiliary working channel, such as to supplement a working channel of a medical scope operatively associated therewith, such as to house actuation instrumentation and/or to allow one or more instruments to extend therethrough. Additionally or alternatively, the lumen may allow fluid to pass therethrough to facilitate irrigation, suction, insufflation, etc.

Various medical devices, including medical scopes, have one or more flexible elongate members and various mechanisms, such as internal pulleys and cables, to steer (e.g., navigate and bend, etc.) the flexible elongate member through a patient's body (including tortuous body passages). For instance, some flexible elongate members may be articulated in one or more planes, such as for up-down movement and/or left-right movement. Articulation devices and systems may be operated to direct flexible elongate members to a desired target site within the patient's body. In some instances, a high degree of flexing, such as to the point of retroflexing (bending backwards) may be desired to access a target site within a patient's body. However, such retroflexion may present various challenges, such as with respect to distortion of the lumen through the retroflexed member. It is with respect to these and other considerations that the present improvements may be useful.

### SUMMARY OF THE DISCLOSURE

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a flexible tubular elongate member is disclosed for use with a retroflexion system. The flexible tubular elongate member includes an exterior surface and an interior surface extending between a proximal end and a distal end of the flexible tubular elongate member, the interior surface defining one or more lumens through the flexible tubular elongate member between the proximal end and the distal end of the flexible tubular elongate member; and a flexion section along the length of the flexible tubular elongate member between the proximal end and the distal end configured to be kink-resistant and/or deformation resistant. In some aspects, at least one of the exterior surface or the interior surface of the flexion section is shaped, configured, and/or dimensioned to facilitate flexing of the flexion section.

In some aspects, at least one of the exterior surface or the interior surface of the flexion section is corrugated. In some aspects, the flexion section is reinforced. In some aspects, the flexion section includes a wire coil between the exterior surface and the interior surface thereof reinforcing the flexion section. In some aspects, the exterior surface and the interior surface of the flexion section are corrugated as a result of the shape of the wire coil therebetween.

In some aspects, the flexion section includes a wire coil between the exterior surface and the interior surface thereof to facilitate flexion of the flexion section. In some aspects, the flexion section further comprises an outer tubular core layer over the wire coil\ and an inner tubular core layer within the wire core, thereby forming a multi-layer wall of the flexion section.

In some aspects, one or more wall regions of the flexion section has alternating thick and thin wall thicknesses.

In some aspects, the cross-sectional shape of the one or more lumens through the flexion section remains substantially the same as the flexion section is flexed.

In some aspects, the flexion section extends along a portion of a distal end of the flexible tubular elongate member.

In accordance with various principles of the present disclosure, a system for retroflexing a flexible elongate member of a medical device includes an elongated actuation component extending along the flexible elongate member and having a proximal end and a distal end; a distal mount configured to be mounted with respect to the distal end of the flexible elongate member; a pivot mount configured to be mounted with respect to the flexible elongate member proximal to the distal mount; and an auxiliary flexible tubular elongate member configured to extend along the flexible elongate member and having a proximal end and a distal end. In some aspects, the distal end of the auxiliary flexible tubular elongate member is operatively associated with the distal mount; the distal end of the actuation component is operatively associated with the distal mount to cause flexion of the distal end of the flexible elongate member when the actuation component is pulled proximally; an actuation region of the actuation component pivots with respect to the pivot mount when the actuation component is pulled proximally to cause the flexible elongate member to retroflex; the auxiliary flexible tubular elongate member comprises a flexion section operatively associated with the distal mount to flex when the actuation component is pulled proximally; the flexion section is configured to be kink-resistant and/or deformation resistant when flexed; and one or more lumens are defined through the flexion section and have a cross-sectional shape unaffected and unchanged by flexing of the flexion section.

In some aspects, the system further includes a flexible tubular elongate member having a proximal end and a distal end with a lumen extending therebetween. In some aspects, the actuation component extends through the flexible tubular elongate member lumen; the flexible tubular elongate member is configured to be mounted with respect to the flexible elongate member with the distal end of the flexible tubular elongate member proximal to the distal end of the flexible elongate member; the distal end of the flexible tubular elongate member forms the pivot mount for the actuation component; and the auxiliary flexible tubular elongate member extends along the flexible tubular elongate member with the flexion section extending distally beyond the distal end of the distal end of the flexible tubular elongate member to be operatively associated with the distal mount. In some aspects, the flexion section of the flexible tubular elongate member does not extend to the proximal end of the flexible tubular elongate member. In some aspects, the flexible tubular elongate member extends through a lumen defined through the flexible tubular elongate member.

In some aspects, the flexion section has an exterior surface and an interior surface, the interior surface defining the lumen through the flexion section; and at least one of the exterior surface or the interior surface of the flexion section is shaped, configured, and/or dimensioned to facilitate flexing of the flexion section.

In some aspects, the flexion section is reinforced with a wire coil.

In accordance with various principles of the present disclosure, a method of forming a kink-resistant and/or deformation-resistant flexion section of a flexible tubular elongate member, insertable transluminally into a patient, includes corrugating at least one of an exterior surface or an interior surface of the flexion section to increase flexibility of the flexion section so that a cross-sectional shape of one or more lumens defined through the flexion section is not changed or affected by flexion of the flexion section.

In some aspects, the method further includes corrugating both the exterior surface and the interior surface of the flexion section. In some aspects, the method further includes reinforcing the flexion section with a wire coil. In some aspects, the method further includes forming the flexion section with an outer tubular core layer over the wire coil to form the exterior corrugated surface of the flexion section, and providing the wire coil over an inner tubular core layer to form the interior corrugated surface of the flexion section.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 is an elevational view of an example of an embodiment of a retroflexion device and system with which an auxiliary flexible tubular elongate member formed in accordance with various principles of the present disclosure may be used.

FIG. 2 is a schematic illustration of a retroflexion device and system as in FIG. 1 subject to distal retroflex as applied by the retroflexion system.

FIG. 2A is a detail view of a flexion section of a flexible tubular elongate member such as illustrated within the broken-line area in FIG. 2.

FIG. 3 is an elevational view of a flexible tubular elongate member with a kink-resistant and/or deformation resistant section formed in accordance with various principles of the present disclosure.

FIG. 3A is a detail view along a section of the flexible tubular elongate member illustrated within the broken-line area in FIG. 3.

FIG. 4 is a cross-sectional view of an example of an embodiment of a flexible tubular elongate member formed in accordance with various principles of the present disclosure.

FIG. 5 is an exploded perspective view of a flexible tubular elongate member such as in FIG. 4.

FIG. 6 is a perspective view of an endoscopic suturing system according an embodiment of the invention.

FIG. 7 is a proximal perspective view of a suturing device of the endoscopic suturing system of FIG. 6.

FIG. 8 is a side elevation view of a suturing device, of indefinite length, of the endoscopic suturing system of FIG. 6.

FIG. 9 is a perspective distal end view of a cap assembly attached at the distal end of an endoscope of the endoscope suturing system.

FIG. 10 is a perspective distal end view of cap assembly of the suturing device relative to an endoscope for the endoscopic suturing system.

FIG. 11 is a cross section view across line XI-XI in FIG. 6.

FIG. 12 is a schematic distal end view of the cap assembly of the suturing device relative to an endoscope for the endoscopic suturing system.

FIG. 13 is a view similar to FIG. 12 with the endoscope rotated into alignment.

FIG. 14 is a view similar to FIG. 10 with the endoscope shown properly oriented and advanced to the distal end of a cap assembly.

FIG. 15 is a schematic illustration of a tape applicator.

FIG. 16 is a schematic illustration of a taping system for securing a sheath of the suturing device relative to the endoscope.

FIG. 17 and FIG. 18 illustrate features of the tape of the tape system.

FIG. 19 and FIG. 20 are schematic illustrations of a retroflex system for the endoscopic system, as applied at the proximal handle of the system.

FIG. 21 is a schematic illustration of a detail of one embodiment of a lock for the retroflex system.

FIG. 22 is a schematic illustration of the endoscopic system subject to distal retroflex as applied by the retroflex system.

FIG. 23 is a schematic illustration of a detail of operation of the lock of FIG. 21.

FIGS. 24-28 illustrate use of the tape applicator to apply tape at the cap assembly to secure the cap assembly relative to the distal end of the endoscope.

FIG. 29 illustrates use of the tape along the sheath to secure the sheath to the endoscope.

FIGS. 30-34 illustrate use of the endoscopic system to endoscopically suture tissue.

FIG. 35 and FIG. 36 illustrate disassembly of the tape from about the cap assembly.

FIG. 37 illustrates disassembly of the sheath from the endoscope.

FIG. 38 and FIG. 39 illustrate examples of alignment of a cap assembly as in FIG. 12 and FIG. 13.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. As understood herein, “corresponding” is intended to convey a relationship between components, parts, elements, etc., configured to interact with or to have another intended relationship with one another. Finally, reference to “at” a location or site is intended to include at and/or about the vicinity of (e.g., along, adjacent, proximate, etc.) such location or site.

In accordance with various principles of the present disclosure, a retroflexion device and system is configured for mounting with respect to a flexible elongate member. At least one flexible tubular elongate member may extend along or alongside the flexible elongate member. The present disclosure provides various improvements to articulation devices and systems which may allow the desired flexing of the flexible elongate member with which the device and system are operatively associated, even to the point of retroflexion (bending backwards).

It will be appreciated that as a portion of a flexible tubular elongate member becomes flexed, the lumen therein is also flexed. As the amount of flexion increases, the generally circular cross section of a lumen of a flexible tubular elongate member (e.g., an open accessory channel) may become elongated/ovalized with the cross-section thereof changing from generally circular to generally elliptical. In some instances, as the degree of flexion of currently commercially available flexible tubular elongate members increase, such as to the point of retroflexion (e.g., beyond 90° from its initial position), the cross-section of the lumen therein will continue to elongate until the flexible tubular elongate member folds over and forms a kink. While elongated or kinked, the cross section of the lumen through the flexible tubular elongate member is distorted. In some instances, the distortion narrows the lumen and reduces the flow capacity through the lumen (e.g., for irrigation, suction, and/or insufflation) and/or increases the amount of friction imposed on any instrument extended therethrough. In some instances, the lumen may be so narrowed as to entirely prevent the passing of an instrument or fluid therethrough. Moreover, once the lumen of the flexible tubular elongate member has formed a kink, a return of the flexible tubular elongate member to its straightened or non-flexed position may not fully restore the original cross-sectional geometry of the lumen therein (e.g., the lumen may be permanently distorted), and the “kink” geometry often persists, causing further use of the lumen to be challenging. It will be appreciated that references herein to resisting kinking and/or distortion are not necessarily limited specifically to kinking and/or distortion and should be understood to include other generally undesired effects of bending of a flexible tubular elongate member, such as on the shape and/or efficacy of the lumen therethrough.

In accordance with various principles of the present disclosure, a retroflexion device and system are operatively associated with a flexible elongate member to articulate as well as to retroflex the flexible elongate member. In some aspects, the flexible elongate member already includes a retroflexion device and/or system (e.g., a steering system for a flexible elongate member in the form of a medical scope), and the retroflexion device and/or system of the present disclosure may be used to supplement and/or to increase the maximum flexion capability of the flexible elongate member. One or more flexible tubular elongate members may be extended along the flexible elongate member and the retroflexion device. In accordance with various principles of the present disclosure, at least a distal portion of the flexible tubular elongate member which may be articulated and retroflexed with the flexible elongate member is reinforced to prevent deformation, kinking, flattening, etc., of the flexible tubular elongate member as well as deformation of the lumen(s) therein.

In some aspects, the flexible elongate member is a medical scope with one or more working channels extending longitudinally therethrough, and flexible tubular elongate member provides one or more auxiliary working channels alongside the one or more working channels of the medical scope. However, principles of the present disclosure are applicable to flexible tubular elongate members associated with a retroflexion device and/or system, regardless of the form or character of the flexible elongate member with which the retroflexion device and/or system are operatively associated with to articulate and/or retroflex. Accordingly, it will be appreciated that references herein to medical scopes are for the sake of convenience only, the present disclosure not being limited by the type of associated device with which a flexible tubular elongate member formed in accordance with principles of the present disclosure is operatively associated.

Various embodiments of devices and/or systems for effecting retroflexion of a medical device, and associated methods, as well as various embodiments of flexible tubular elongate members usable therewith and resistant to deformation of the lumen(s) therein, and various methods associated therewith, will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

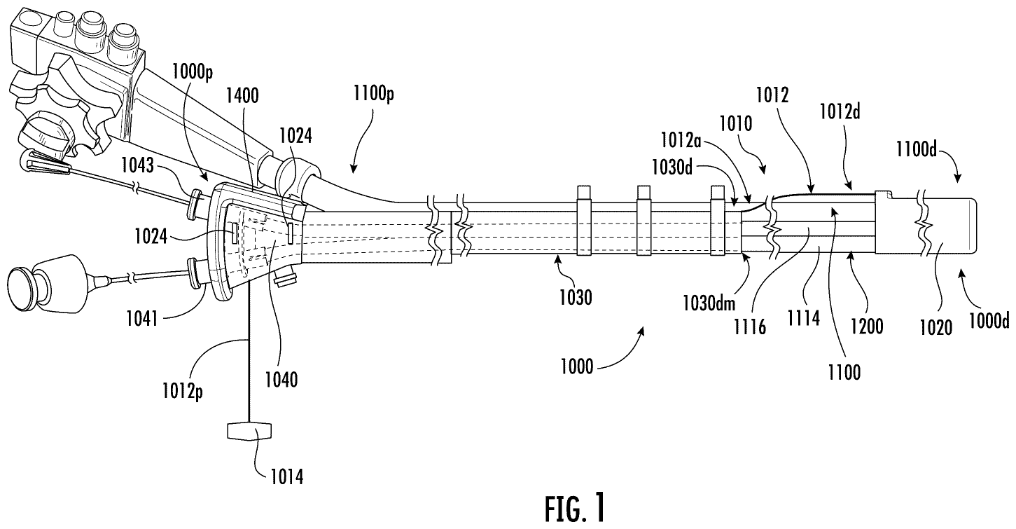

Turning now to the drawings, an example of an embodiment of a retroflexion system 1000 formed in accordance with various principles of the present disclosure is illustrated in FIG. 1. The illustrated example of an embodiment of a retroflexion system 1000 includes a retroflexion device 1010 operatively associated with a flexible elongate member 1100. The flexible elongate member 1100 may be a component of a medical device, or may reference a medical device in general, such as a medical scope, reference being made simply to a flexible elongate member in a non-limiting manner, as the present disclosure need not be limited to a particular flexible elongate member or medical device to be articulated and/or retroflexed by a retroflexion device 1010 or retroflexion system 1000 formed in accordance with various principles of the present disclosure.

The retroflexion device 1010 includes an actuation component 1012 which may be referenced herein, for the sake of convenience and without intent to limit, as a pull string 1012. The pull string 1012 is an elongate element having sufficient strength to be actuated from a proximal end 1012p thereof to cause a distal end 1012d thereof to actuate the flexible elongate member 1100 with which the retroflexion device 1010 is operatively associated. The pull string 1012 may be formed of a textile, polymer, or metal monofilament, multifilament, cable-stranded element, etc., having the appropriate strength, such as selected with reference to the longitudinal extent thereof, the flexibility of the flexible elongate member 1100, etc. Articulation and/or retroflexion of the flexible elongate member 1100 by the retroflexion system 1000 of the present disclosure may advantageously direct a distal working end, or the opening of a working channel (of a medical scope) of the flexible elongate member 1100 to a target site within a patient's body. The working end of a medical device (either the flexible elongate member 1100, if in the form of a medical device, or a medical device extending through a working channel of a flexible elongate member in the form of a medical scope) may thereby be directed more accurately to the target site than previously achievable by prior art systems and devices.

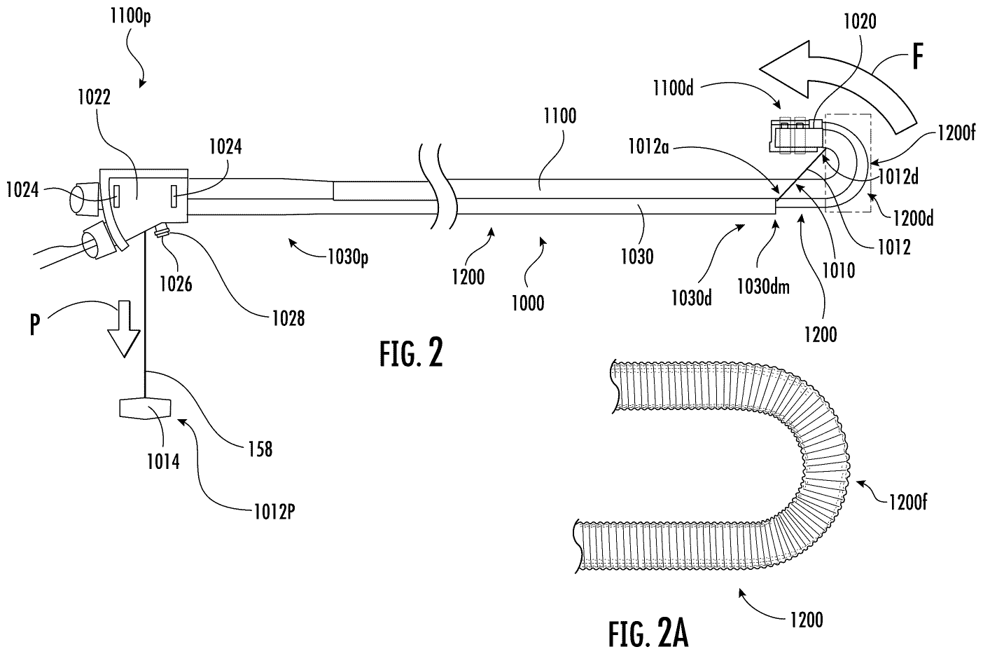

In accordance with various principles of the present disclosure, the distal end 1012d of the pull string 1012 is operatively associated with the flexible elongate member 1100 adjacent a section of the flexible elongate member 1100 which is to be articulated by the retroflexion system 1000. For maximum efficacy, the distal end 1012d of the pull string 1012 may be positioned at and/or operatively associated with the distalmost, terminal end of the flexible elongate member 1100. However, it will be appreciated that the distal end 1012d of the pull string 1012 need not be positioned exactly at the distalmost, terminal end of the distal end 1100d of the flexible elongate member 1100, but, instead, may be adjacent, and somewhat proximal to, the distalmost, terminal end of the device to be articulated. The exact position may be determined by one of ordinary skill in the art, taking into account such factors as the flexibility of the device to be articulated, the anchored region of the pull string to allow for flexing, etc. The pull string 1012 may be operatively associated with the flexible elongate member 1100 in any of a variety of manners, the present disclosure not being limited in this regard. For example, the retroflexion system 1000 may include one or more mounts configured to operatively associate the retroflexion device 1010 with the flexible elongate member 1100. In the example of an embodiment illustrated in FIG. 1, the retroflexion system 1000 includes a distal mount 1020 having a section thereof configured to be engaged with the flexible elongate member 1100. For instance, the distal mount 1020 may include a concave section sized, shaped, configured, and/or dimensioned to receive a section of the flexible elongate member 1100. The distal mount 1020 may extend at least 180°, and in some embodiments, greater than 180°, about the circumference of the flexible elongate member 1100, such as to be maintained engaged with, mounted on, etc., the flexible elongate member 1100 (e.g., by friction or interference fit). Alternatively, the distal mount 1020 may be a collar which may encircle and be affixed to (tightened, adhered, clamped, etc., with respect to) the flexible elongate member 1100. Other configurations or forms of mounts are within the scope and spirit of the present disclosure, the present disclosure not being limited in this regard. The distal end 1012d of the pull string 1012 is operatively associated, such as coupled (e.g., fixedly coupled), with respect to the distal mount 1020 so that when the pull string 1012 is pulled proximally (e.g., by pulling on a proximal end 1012p thereof), the distal end 1012d of the pull string 1012 pulls the distal mount 1020 to flex the flexible elongate member 1100 proximally (e.g., in the direction of flexion arrow F), as illustrated in FIG. 2.

In order to cause flexing of the flexible elongate member 1100, an actuation region 1012a of the pull string 1012 proximal to the distal end 1012d thereof is held against movement (e.g., anchored) with respect to the flexible elongate member 1100 (while still being longitudinally movable along the flexible elongate member 1100). The actuation region 1012a may provide a pivot point for the pull string 1012 to flex the flexible elongate member 1100. The distance between the actuation region 1012a and the distal end 1012d of the pull string 1012 may be selected to achieve a desired bend radius. As may be appreciated by those of ordinary skill in the art, the materials of the pull string 1012 and/or the flexible elongate member 1100 may affect selection of such distance to achieve the desired articulation of the flexible elongate member 1100. It will be further appreciated that the actuation region 1012a may be positioned independently of any of the lumens/working channels through the system 1000.

In some aspects, the actuation region 1012a of the pull string 1012 is held against lateral movement with respect to the flexible elongate member 1100 and the distal mount 1020 with the aid of a pivot mount for the actuation region 1012a. Such pivot mount may be similar to the above-described distal mount 1020 and thus reference is made to the above description for the sake of brevity and without intent to limit. In some aspects, the pivot mount which holds the actuation region 1012a of the pull string 1012 laterally with respect to the flexible elongate member 1100 may be the distal end 1030d of a flexible tubular elongate member 1030 (e.g., a sheath) extending along the flexible elongate member 1100 and defining a lumen therethrough through which the pull string 1012 translates/movably extends. The flexible tubular elongate member 1030 extends generally from a proximal end 1100p of the flexible elongate member 1100 towards a distal end 1100d of the flexible elongate member 1100.

To allow operation of the pull string 1012 and to provide an extent along which pulling of the pull string 1012 with respect to the flexible elongate member 1100 causes flexing of a distal flexing section of the flexible elongate member 1100, the actuation region 1012a of the pull string 1012 is spaced proximally from the distal end 1012d of the pull string 1012. In some aspects, the actuation region 1012a may be considered to be determined by the position of the pivot mount for the actuation region 1012a. For instance, a pivot mount configured similar to the distal mount 1020 is proximally spaced from the location at which the distal end 1012d of the pull string 1012 is operatively associated with the flexible elongate member 1100 (e.g., proximally spaced from the distal mount 1020 with which the pull string 1012 is operatively associated) to space the actuation region 1012a of the pull string 1012 proximally from the distal end 1012d of the pull string 1012 to achieve the desired flexion of the pull string 1012. In embodiments in which the pivot mount which holds the actuation region 1012a of the pull string 1012 is defined by the distal end 1030d of a flexible tubular elongate member 1030, the flexible tubular elongate member 1030 does not extend along the entire length of the flexible elongate member 1100. As illustrated in FIG. 1 and FIG. 2, the distalmost end 1030dm of the example of an embodiment of a flexible tubular elongate member 1030 is positioned proximal to the distal end 1100d of the flexible elongate member 1100. The pull string 1012 extends distally out of the flexible tubular elongate member 1030 and distal to the distalmost end 1030dm of the flexible tubular elongate member 1030 to be operatively associated with the distal end 1100d of the flexible elongate member 1100. As such, in some aspects, the distalmost end 1030dm of the flexible tubular elongate member 1030 may provide an anchor or pivot mount/pivot point for the actuation region 1012a of the pull string 1012, such as by serving as a pivot mount. It will be appreciated that the distance between the distal end 1012d of the pull string 1012 and the actuation region 1012a may influence the bending radius of the flexed region of the flexible elongate member 1100. In some embodiments, such as illustrated in FIG. 1 and FIG. 2, the pull string 1012 is operatively associated with an adaptor/proximal mount 1040 provided along a proximal end 1000p of the retroflexion system 1000, such as along a proximal end 1030p of the flexible tubular elongate member 1030 along which the pull string 1012 of the retroflexion device 1010 extends.

As may be appreciated, retroflexion devices and/or systems of the present disclosure may be operatively associated with one or more devices, such as devices used in conjunction with and/or independently of the flexible elongate member 1100, such as to achieve further articulation, additional flexibility, and/or reach, optionally independently of other devices of the system. In some embodiments, one or more auxiliary flexible tubular elongate members 1200 may extend generally longitudinally along/alongside the flexible elongate member 1100 (e.g., along/alongside the exterior or outer surface of the flexible elongate member 1100) with which the retroflexion system 1000 and/or device 1010 is associated, such as to provide one or more auxiliary working channels to the overall system. In some aspects, the distal end 1200d of the auxiliary flexible tubular elongate member 1200 is operatively associated with the distal mount 1020 of the retroflexion system 1000 (e.g., coupled thereto) so that the auxiliary flexible tubular elongate member 1200 is retroflexed in a manner similar to the retroflexion of the flexible elongate member 1100 described above. In some aspects, the proximal mount 1040 is configured to facilitate advancement of devices through lumens defined within the flexible tubular elongate member 1030, such as by providing ports 1041, 1043 facilitating access to the lumens within the flexible tubular elongate member 1030. In some embodiments, a retroflexion system 1000 formed in accordance with various principles of the present disclosure may be operatively associated with a flexible elongate member 1100 with one or more working channels defined/extending therethrough. In some aspects, each of the one or more auxiliary flexible tubular elongate members 1200 has at least one auxiliary working channel defined therethrough. The auxiliary working channels of the auxiliary flexible tubular elongate members 1200 augment the working channels of the overall system (e.g., provide working channels in addition to working channels provided by the flexible elongate member 1100). Additional devices may be extended through an auxiliary working channel defined through an auxiliary flexible tubular elongate member 1200.

In accordance with various principles of the present disclosure, a retroflexion system 1000 formed in accordance with various principles of the present disclosure may articulate, flex, and/or retroflex a flexible tubular elongate member operatively associated therewith to an extent which may cause the lumen through the flexible tubular elongate member to elongate or ovalize (e.g., if circular in a neutral elongated configuration of the flexible tubular elongate member) or even become distorted. If the lumen is distorted, then it may not properly function as a working channel through which further instruments or materials may effectively be delivered, withdrawn, etc. In worse case scenarios, flexing or retroflexing the flexible tubular elongate member may cause permanent distortion of the lumen. For instance, flexing of a flexible tubular elongate member greater than about 90°, such as greater than about 145°, such as greater than about 180°, and up to or even greater than about 210° (including various increments therebetween, such as increments of 1°) may distort the lumen therein. The same concerns apply to lumens of auxiliary flexible tubular elongate members 1200 used with the system 1000.

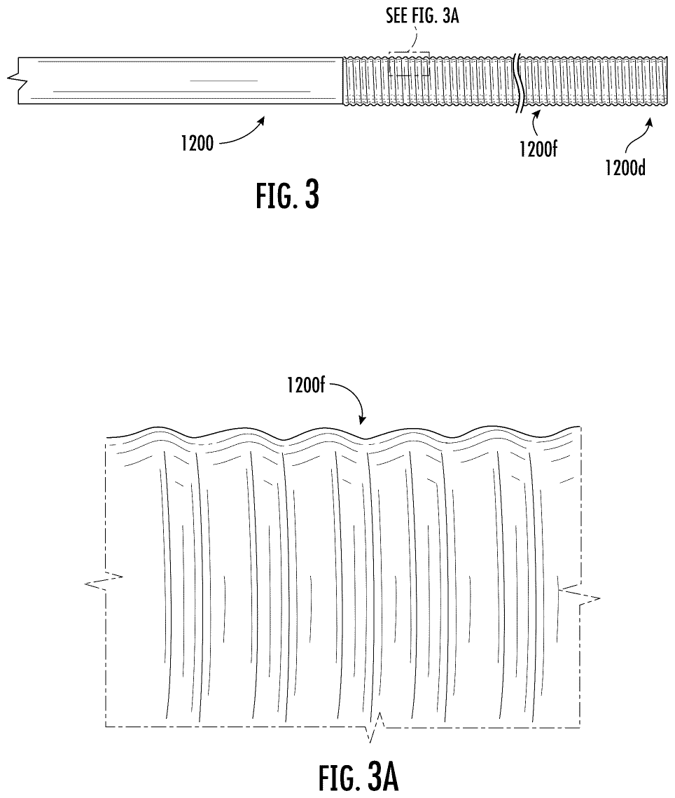

In accordance with various principles of the present disclosure, a flexible tubular elongate member which is operatively associated with a retroflexion system and/or device, such as those disclosed herein, is shaped, configured, and/or dimensioned to resist kinking or otherwise bending or flexing in a manner which may distort or otherwise affect the shape and/or functionality of the lumen therein. It will be appreciated that reference is made herein to resisting kinking and/or distortion for the sake of convenience and without intent to limit. In some aspects, at least a section of the flexible tubular elongate member (e.g., a feature of the wall of the flexible tubular elongate member) is shaped, configured, dimensioned, etc., to facilitate increased bending without kinking and without distorting the lumen defined therein. In some aspects, the material of the flexible tubular elongate member is reinforced. In some aspects, the shape, configuration, dimensions, etc., of the flexible tubular elongate member are modified by the addition of a reinforcing material and/or structure to the wall of the flexible tubular elongate member. It will be appreciated that references to improvements to flexible tubular elongate members are intended to include auxiliary flexible tubular elongate members such as described herein, and references to “auxiliary” flexible tubular elongate members are applicable to flexible tubular elongate members in general unless otherwise stated. In some embodiments of flexible tubular elongate member(s) formed in accordance with various principles of the present disclosure to resist deforming/kinking, a section of the flexible tubular elongate member is configured to facilitate flexing of such section of the flexible tubular elongate member without kinking and/or deforming. In the example of an embodiment of an auxiliary flexible tubular elongate member 1200 illustrated in FIG. 2, and on its own in FIG. 3, only a flexion section 1200f of the auxiliary flexible tubular elongate member 1200 (i.e., not the full longitudinal extent/length of) is kink-resistant and/or deformation resistant. In the illustrated example of an embodiment, the flexion section 1200f is along the distal end 1200d of the auxiliary flexible tubular elongate member 1200. However, the present disclosure need not be limited in this regard.

The kink-resistant and/or distortion resistant flexion section of a flexible tubular elongate member formed in accordance with various principles of the present disclosure may be formed in a variety of manners to result in the desired resistance to kinking and/or distortion. In some aspects, at least a section of the flexible tubular elongate member may be configured to be multi-jointed to facilitate flexing thereof. In some embodiments, one or both of the surfaces of at least a section of a flexible tubular elongate member (e.g., the exterior surface, or the interior surface such as the surface defining the lumen of the flexible tubular elongate member) and/or the cross-sectional shape of the flexible tubular elongate member is shaped, configured, and/or dimensioned to facilitate flexing of such section without kinking and/or deforming. In some embodiments, a section of a flexible tubular elongate member formed in accordance with various principles of the present disclosure is corrugated, or otherwise shaped to not be straight or flat, to facilitate flexing thereof. It will be appreciated that the configuration of a section of the flexible tubular elongate member which facilitates flexing without kinking may be described as corrugated, concertinaed, grooved, otherwise not straight/flat etc., one of ordinary skill in the art being capable of understanding the nature of section in view of the following description. In some aspects, when viewed laterally, and/or in cross-section, a series of ridges may be formed on a surface (exterior and/or interior) of a portion of the flexible tubular elongate member. In some aspects, the wall of a kink-resistant and/or deformation-resistant flexible tubular elongate member formed in accordance with various principles of the present disclosure may have alternating sections of thin and thick wall thicknesses, such as circumferentially-extending regions or sections along the longitudinal axis of the flexible tubular elongate member 1200 alternating in thickness. In other words, the wall of the kink-resistant and/or deformation-resistant flexible tubular elongate member may include one or more wall regions thinner than immediately proximal and immediately distal wall regions. A wall of a flexible tubular elongate member formed in accordance with various principles of the present disclosure with ridges or corrugations or the like may facilitate flexing, such as by making the wall more elastic and/or by reducing stresses on the wall such as at the thinner regions thereof (which more readily flex than do the thicker regions) and/or by stretching or otherwise accommodating differences in inner and outer curvature of a bent portion of the flexible tubular elongate member.

An example of an embodiment of a kink-resistant and/or deformation-resistant flexible tubular elongate member is illustrated in the detail view FIG. 2A showing the distal portion 1200d of the auxiliary flexible tubular elongate member 1200 of FIG. 2. As may be appreciated with reference to FIG. 2A, at least the exterior surface (the visible, illustrated outer surface) of the illustrated section is corrugated. As the flexible tubular elongate member 1200 is flexed, the geometry of the ridges (or other corrugated shape) allows for a greater degree of flexion while maintaining the cross-sectional, typically circular, geometry of the flexible tubular elongate member 1200. In some aspects, the shape/configuration of the at least exterior surface of the section of the flexible tubular elongate member 1200 illustrated in FIG. 2A is similar to a bellows or an accordion, allowing the interior radius of the flexed flexible tubular elongate member 1200 to collapse and the outer radius of the flexed flexible tubular elongate member 1200 to expand. An example of an embodiment of such configuration of at least the exterior surface of a flexion section 1200f of the example of an auxiliary flexible tubular elongate member 1200 illustrated in FIG. 3 is illustrated in a detail view provided in FIG. 3A. As may be appreciated, a flexible tubular elongate member with corrugations, or the like, allows greater flexibility without kinking and without distortion of the lumen therein than may be achieved by prior art flexible tubular elongate members.

As used in conjunction with a retroflexion system 1000 and/or device 1010 as described herein, such as illustrated in FIG. 1 and FIG. 2, an auxiliary flexible tubular elongate member 1200 formed in accordance with various principles of the present disclosure allows delivery of a device or materials through the lumen thereof to extend not only in a linear direction from the auxiliary flexible tubular elongate member 1200 and with respect to the lumen thereof, but also at various angles with respect to a more proximal portion of the auxiliary flexible tubular elongate member 1200 (a portion extending proximal to the flexed section of the auxiliary flexible tubular elongate member 1200). More particularly, a device or materials delivered through a kink-resistant and/or distortion-resistant auxiliary flexible tubular elongate member 1200 formed in accordance with various principles of the present disclosure may extend greater than about 90° from an initial distally-extending direction of the auxiliary flexible tubular elongate member 1200 and in a direction towards the proximal end of the auxiliary flexible tubular elongate member 1200, such as illustrated in FIG. 2. As may be appreciated, flexion of a flexion section 1200f of a kink-resistant and/or distortion-resistant auxiliary flexible tubular elongate member 1200 from an initially distal direction (generally aligned with a more proximally-extending section thereof) to a direction towards the proximal end 1200p of the auxiliary flexible tubular elongate member 1200 does not distort or otherwise impede delivery of devices and/or materials through the distal flexion section 1200f, as may be appreciated with reference to FIG. 2A. In view of the above, a retroflexion system 1000 and/or device 1010 formed in accordance with various principles of the present disclosure may flex the distal flexion section 1200f of the kink-resistant and/or distortion-resistant auxiliary flexible tubular elongate member 1200 greater than about 90°, such as greater than about 145°, such as greater than about 180°, and up to or even greater than about 210° (including various increments therebetween, such as increments of 1°) from an initial position extending in a distal direction with respect to the system 1000/device 1010 (and generally from an initial position axially aligned with the proximal portion of the auxiliary flexible tubular elongate member 1200). Further in view of the above, the kink-resistant and/or distortion-resistant wall of the auxiliary flexible tubular elongate member 1200 allows passage of a device or materials through the lumen thereof, without the flexion of the auxiliary flexible tubular elongate member 1200 affecting delivery and/or movement of the device or materials.

It will be appreciated that the formation of corrugations along a flexible tubular elongate member, such as by providing wall sections thinner than immediately adjacent sections proximal and distal thereto, may result in loss of column strength and/or other weakening of the flexible tubular elongate member. In some embodiments, the wall of a kink-resistant and/or deformation-resistant flexible tubular elongate member formed in accordance with various principles of the present disclosure is reinforced. In some embodiments, the kink-resistant and/or deformation-resistant flexible tubular elongate member is reinforced with a filament, wire, braid, mesh or the like, which is more rigid than the material from which the tubular elongate member is formed. For instance, the kink-resistant and/or deformation-resistant flexible tubular elongate member may have a tubular wall formed of a flexible, resilient, biocompatible material (e.g., a polymer such as a thermoplastic elastomer, or PEBAX® block copolymers made up of rigid polyamide and soft polyether blocks), and the filament may be formed of a more rigid material, such as a metal. In some embodiments, the filament is a coiled metal wire extending through the wall of the kink-resistant and/or deformation-resistant flexible tubular elongate member formed in accordance with various principles of the present disclosure. In some aspects, the filament extends through thicker parts of a corrugated wall. In some aspects, the filament contributes to the increased thickness of the wall. In some embodiments, the filament is a rounded wire or flat wire. If the filament is braided or in a mesh configuration, the pattern of the braid may be a 1:1, 1:2, 2:2, etc., pitch of wires, such as which may be selected and/or controlled to impart various levels of reinforcement.

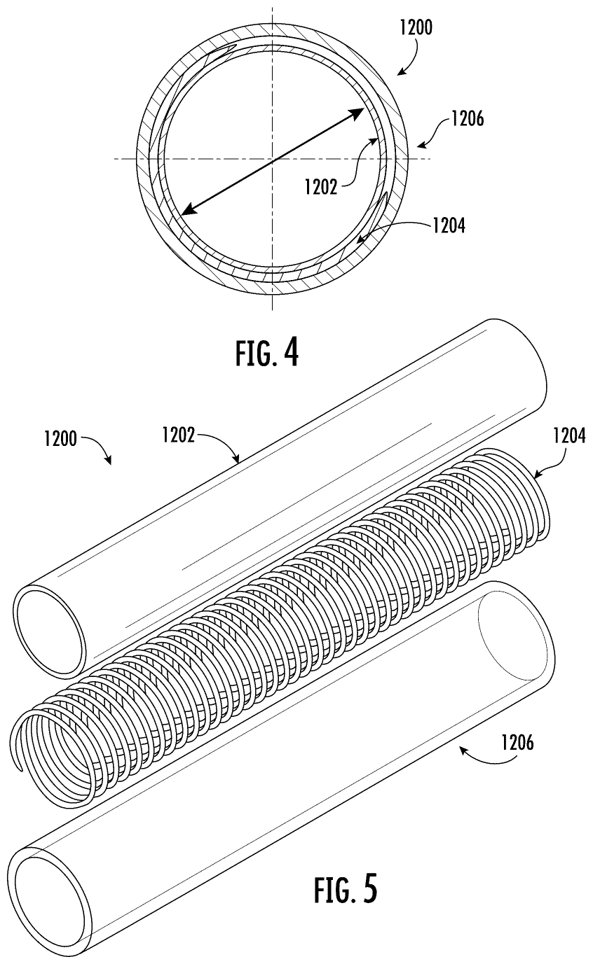

In some aspects, a polymeric tubular wall is formed over a coiled wire to form helical grooves in the resultant flexible tubular elongate member that follow the exterior geometry of the coiled wire, thereby forming a kink-resistant and/or deformation-resistant flexible tubular elongate member in accordance with various principles of the present disclosure. In some aspects, a kink-resistant and/or deformation-resistant flexible tubular elongate member 1200 of the present disclosure is constructed from an inner tubular core layer, a coiled wire disposed over the inner tubular core layer, and an outer tubular core layer disposed over the coiled wire, such as illustrated in FIG. 4 (showing a cross-section through an example of an embodiment of a multi-layer wall of a kink-resistant and/or deformation resistant flexible tubular elongate member formed in accordance with various principles of the present disclosure). An exploded view of an example of an embodiment of a multi-layer kink-resistant and/or deformation-resistant flexible tubular elongate member 1200 formed in accordance with various principles of the present disclosure is illustrated in FIG. 5. The inner layer (which may also be referenced as an inner core), the coiled wire, and the outer layer (which may also be referenced as an outer core) may be joined together in any of a variety of manners to achieve a strong bond therebetween. For instance, the materials of at least the inner layer and the outer layer may be fusable together (e.g., through the spacings between coils of the wire positioned therebetween). In some aspects, various parameters of the reinforcement coil (e.g., spacing between coils/pitch; wire diameter; wire shape such as cross-sectional shape (round vs. flat vs. square); etc.) may be tuned to the particular needs of the system in which the kink-resistant and/or deformation-resistant flexible tubular elongate member is to be used. Additionally or alternatively, the fill of the inner layer between the coil spacing, and/or the outer layer between the coil spacing, may be tuned to achieve the desired corrugated effect on the outer diameter and/or inner diameter of the kink-resistant and/or deformation-resistant flexible tubular elongate member. Following fusion of the inner core, the coiled wire, and the outer core, the external surface of the reinforced catheter has a helical groove that follows the exterior geometry of the coiled wire, such as illustrated in FIG. 3A. It should be appreciated that the configuration illustrated in FIG. 3A need not be limited to resulting from such formation of a kink-resistant and/or deformation resistant flexible tubular elongate member.

As described above with reference to the example of an embodiment of a retroflexion system 1000 and retroflexion device 1010 illustrated in FIG. 2, the distalmost end 1030dm of the flexible tubular elongate member 1030 of the illustrated retroflexion system 1000 is spaced proximally from the distal mount 1020 with which the distal end 1012d of the pull string 1012 is operatively associated. In some embodiments, the flexible tubular elongate member 1030 includes one or more lumens therethrough through which an auxiliary flexible tubular elongate member 1200 may extend. It may be desirable to extend the distal end 1200d of the auxiliary flexible tubular elongate member 1200 to substantially the same location as the distal end 1100d of the flexible elongate member 1100 (e.g., to extend instruments to a target site at which the flexible elongate member 1100 is to be positioned). Because the flexible tubular elongate member 1030 is shorter than the flexible elongate member 1100 along which it extends, the auxiliary flexible tubular elongate member 1200 must extend beyond the distalmost end 1030dm of the flexible tubular elongate member 1030 to reach the distal end 1100d of the system 1000. It is noted that although the auxiliary flexible tubular elongate member 1200 is illustrated as exiting the flexible tubular elongate member 1030 at substantially the same axial location at which the pull string 1012 exits and/or at substantially the same location as the pivot mount for the actuation region 1012a of the pull string 1012, the axial location at which the auxiliary flexible tubular elongate member 1200 exits the flexible tubular elongate member 1030 may be proximal or distal to the pivot mount for the actuation region 1012a of the pull string 1012, the present disclosure not being limited in this regard. The flexible tubular elongate member 1030 may provide sufficient reinforcement for the portion of the auxiliary flexible tubular elongate member 1200 extending therethrough. Thus, the portion of the auxiliary flexible tubular elongate member 1200 extending through the flexible tubular elongate member 1030 need not have additional reinforcement as may be provided in the portion extending outside and/or distally beyond the flexible tubular elongate member 1030. In some aspects, the portion of the auxiliary flexible tubular elongate member 1200 extending within the flexible tubular elongate member 1030 is constructed using standard materials, such as a thermoplastic elastomer extruded to form the desired geometry including forming one or more open lumens. The one or more lumens may have a generally circular cross-section. In some aspects, each lumen of the flexible tubular elongate member 1030 may be individually reinforced in lieu of or in addition to the flexible tubular elongate member 1030 or the auxiliary flexible tubular elongate member 1200.

The distal portion 1200d of the auxiliary flexible tubular elongate member 1200 which extends beyond the distalmost end 1030dm of the flexible tubular elongate member 1030 is the portion which is retroflexed by the retroflexion system 1000 and device 1010. Thus, in some aspects, only the distal portion 1200d of the auxiliary flexible tubular elongate member 1200 which extends beyond the distalmost end 1030dm of the flexible tubular elongate member 1030 is reinforced or otherwise formed to resist kinking and/or deformation in accordance with various principles of the present disclosure. In other words, the flexion section 1200f of the auxiliary flexible tubular elongate member 1200 may be provided at least (and, in some aspects, only) along the section of the auxiliary flexible tubular elongate member 1200 extending distal to the distalmost end 1030dm of the flexible tubular elongate member 1030.

The kink-resistant and/or deformation resistant distal flexion section 1200f may be bonded, fused, or otherwise coupled to (preferably fixedly connected with) the proximal portion of the auxiliary flexible tubular elongate member 1200. The proximal (non-reinforced) portion of the kink-resistant and/or deformation-resistant flexible tubular elongate member and the distal portion of the kink-resistant and/or deformation-resistant flexible tubular elongate member may be joined together in any manner known to those of ordinary skill in the art to achieve the desired bonding connection to remain coupled during normal use of the kink-resistant and/or deformation-resistant flexible tubular elongate member.

The length of the distal flexion section 1200f may be selected based on the distance between the distalmost end 1030dm of the flexible tubular elongate member 1030 and the distal end 1000d of the system 1000 (e.g., the location of the distal mount 1002 and/or the distal end 1100d of the flexible elongate member 1100). In embodiments in which the flexible elongate member 1100 is a medical scope, the length of the distal flexion section 1200f may correspond to the length of a distal “bending section” of a standard medical scope. Because the length of a kink-resistant and/or deformation-resistant flexible tubular elongate member may be more costly to manufacture than the more typical proximal section of the kink-resistant and/or deformation-resistant flexible tubular elongate member, the overall cost of the kink-resistant and/or deformation-resistant flexible tubular elongate member may be reduced by forming only the portions thereof that experience the most flexing/tortuosity to be kink-resistant and/or deformation-resistant.

As may be appreciated, a kink-resistant and/or deformation-resistant flexible tubular elongate member formed in accordance with various principles of the present disclosure is configured to allow unimpeded movement of a device or material through the lumen thereof even at extreme degrees of flexion. In accordance with various principles of the present disclosure, to further facilitate case of movement of devices or materials through the lumen of a kink-resistant and/or deformation-resistant flexible tubular elongate member, the lumen may be coated and/or infused and/or formed with a reduced-friction and/or lubricious material. In some aspects, such material is limited to the distal flexion section of the kink-resistant and/or deformation-resistant flexible tubular elongate member. However, it will be appreciated that such material may be provided along the entire length of the kink-resistant and/or deformation-resistant flexible tubular elongate member if desired. In some aspects, additives may be included in the material of the auxiliary flexible tubular elongate member 1200 for one or more purposes (e.g., reduction of friction, such as between the lumen within the auxiliary flexible tubular elongate member 1200 and another element extending therethrough). For instance, one or more additives may be compounded/mixed with various elastomers (e.g., block copolymers, such as made up of rigid polyamide blocks and soft polyether blocks, such as raw material PEBAX®) to achieve the desired properties, such as which may improve functionality of the auxiliary flexible tubular elongate member, the kink-resistant and/or deformation-resistant flexible tubular elongate member, and/or the system in general. In some aspects, the material is a polytetrafluoroethylene (PTFE) material, as 2μ particulates dispersed in or coated over the material from which the tubular wall of the kink-resistant and/or deformation-resistant flexible tubular elongate member is formed. In some instances, the material is flushed through the lumen of the kink-resistant and/or deformation-resistant flexible tubular elongate member. In some aspects, the material which is flushed through the lumen includes a solvent which leaves behind a thin layer of reduced-friction and/or lubricious material such as PTFE. Additionally or alternatively, a low-friction inner liner (e.g., formed of PTFE) could be added, such as in the form of a flush (e.g., a PTFE flush, or another PTFE-like solvent-carried solutions which can be flushed), and/or as a layer fused to the inner/interior surface of the lumen defined through the auxiliary flexible tubular elongate member 1200. In some aspects, the lumens of the flexible tubular elongate member 1200 may be otherwise reinforced to allow for incremental or maximum flexion without kinking. It will be appreciated that various other lumens of the system 1000 described herein may benefit from such treatments as well.

It may be appreciated that the proximal section of the kink-resistant and/or deformation-resistant flexible tubular elongate member, which may not be as kink-resistant or deformation-resistant as the distal flexion section, may have a friction-reducing additive or y coating through the lumen extending therein, or any other treatment such as described with respect to the distal section of the kink-resistant and/or deformation-resistant flexible tubular elongate member. The reduced-friction and/or lubricious material within the lumen of the distal flexion section may reduce friction or provide even further lubriciousness in view of the extreme flexion thereof and resulting flexion of the lumen thereof.

Although the above disclosure is with reference to a flexible tubular elongate member used in conjunction with a retroflexion device and/or system, principles of the present disclosure may implemented in or applied to any flexible tubular elongate member with lumens or channels therein which are subjected to high degrees of flexion, such as up to and exceeding retroflexion of 180 degrees or greater.

In view of the above, it may be appreciated that a retroflexion system 1000 formed in accordance with various principles of the present disclosure may be used in conjunction with an articulation mechanism of the flexible elongate member 1100 with which the retroflexion system 1000 is operated. In such manner, the retroflexion system 1000 operates as an assist to the existing system of the flexible elongate member 1100, such as to normalize the articulation functionality of the pre-existing system of the flexible elongate member 1100. The retroflexion system 1000 of the present disclosure ensures that the desired degree of retroflexion is achieved for the procedure to be performed with the use of the flexible elongate member 1100. In some aspects, a retroflexion system 1000 formed in accordance with various principles of the present disclosure to be used in conjunction with an existing device or system, and mounted with respect to such device or system, facilitates articulation. For instance, pulling of an actuation device, such as the pull string 1012 of the above-described example of an embodiment of a retroflexion system 1000, extending along/alongside the exterior/outside of the device or system to be actuated applies a tangential force which may apply a greater force to the device or system than a force applied by an actuation element extending therethrough (within, rather than outside, the device/system). Moreover, the retroflexion system 1000 may be applied to various auxiliary devices, such as auxiliary flexible tubular elongate members 1200 extended along the flexible elongate member 1100. A retroflexion system 1000 such as described herein allows for a greater degree or extent of retroflexion than previously achievable. And, a kink-resistant and/or deformation-resistant flexible tubular elongate member formed in accordance with various principles of the present disclosure is usable with such retroflexion system 1000 without kinking, deforming, distorting, or otherwise not functioning as intended.

Various principles of the present disclosure such as those described above may be implemented in a variety of manners and/or applied to a variety of medical devices, examples of which will now be described. It will be appreciated that various methods described with respect to the described examples of embodiments may be applied to other embodiments, such as described more broadly/generically above.

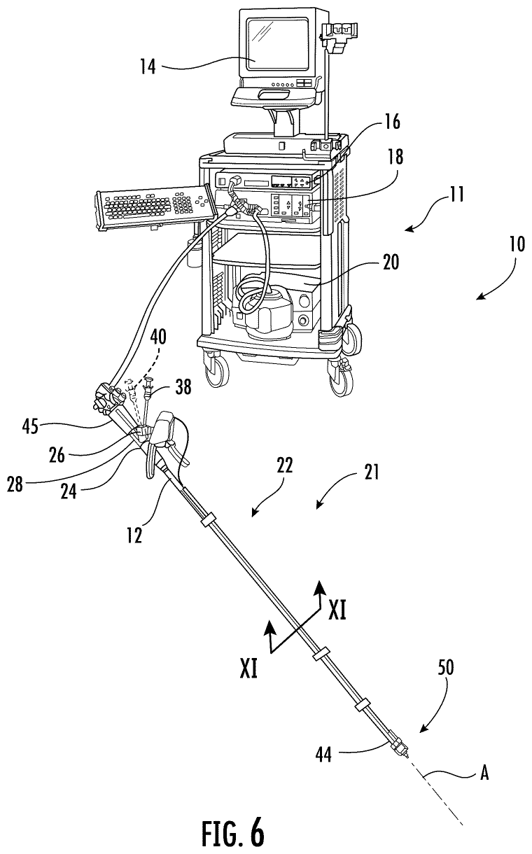

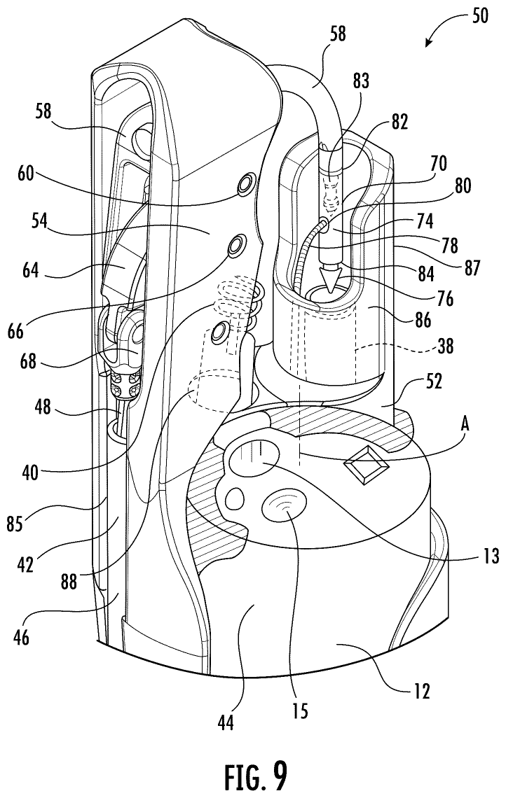

Referring to FIG. 6 through FIG. 9, an endoscopic treatment system 10 includes an endoscope system 11 and an endoscopic suturing system 21. The endoscope system 11 includes an endoscope 12, a video display unit 14, an image processing device 16, a light source 18, and a suction device 20. In accordance with an embodiment, the endoscope 12 has a small profile, generally 5-10 mm in diameter. However, the size of the endoscope is not critical, and elements described herein can be adapted for endoscopes of other sizes. In the example of an embodiment shown, the endoscope 12 has a single instrument channel 13 and an optical lens 15 (FIG. 9). However, the endoscope 12 may have more than one instrument channel or no instrument channel all, as operation of the system does not necessarily require use of the instrument channel through the endoscope. The endoscope 12 includes a distal end 44 and a proximal end 45 and a longitudinal axis A extending therebetween.

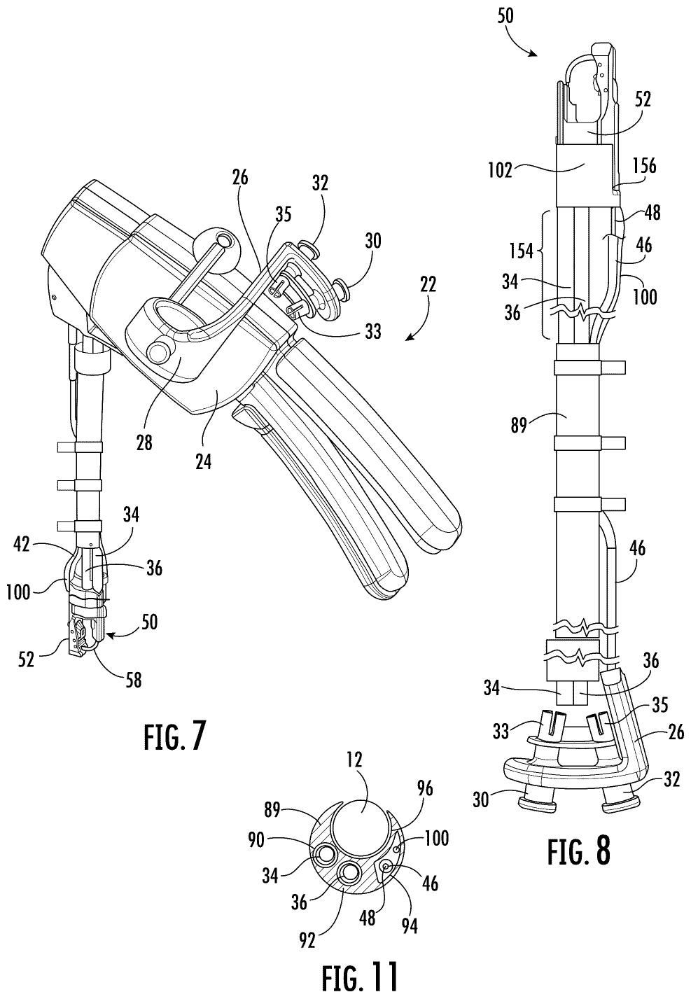

The suturing system 21 includes a suturing device 22 (FIG. 7), a needle assembly 70 (FIG. 9) movable through tissue by the suturing device 22, and first and second devices 38, 40 used in association with the suturing device 22 (FIG. 9).

Referring to FIG. 6, FIG. 7, and FIG. 8, the suturing device 22 has a proximal operable handle 24 provided with a mounting bracket 26 and a collar 28 at which the handle is removably coupled to endoscope 12. The bracket 26 includes first and second instrument ports 30, 32 at which instruments can be received into first and second catheters 34, 36, respectively. First and second tubular connectors 33, 35 are aligned with the ports 30, 32 that couple the ports 30, 32 to the first and second catheters 34, 36.

A transmission assembly 42 includes a transmission sheath 46 and a transmission cable 48 displaceable within the transmission sheath 46, both coupled relative to the handle 24. The transmission sheath 46 is coupled relative to a first portion of the handle (i.e., a stationary member), and the transmission cable 48 is coupled to a second portion of the handle (i.e., a movable lever), such that when the handle 24 is operated the cable 48 is displaced within the transmission sheath 46.

The first and second catheters 34, 36 and the transmission assembly 42 extend from the proximal handle 24, along the outside of the endoscope 12, to a distal cap assembly 50. The distal cap assembly 50 is adapted to be mounted at the distal end 44 of the endoscope 12, and the handle 24 remotely operates the cap assembly 50 via the transmission assembly 42.

Referring to FIG. 9, the cap assembly 50 includes a mount 52, U-shaped support bracket 54 extending distally from the mount, and a needle arm 58 rotatably mounted on the bracket 54 with a first pin 60. A bell crank 64 is rotatably mounted at a second pin 66 on the support bracket 54 and engages the needle arm 58 at intermeshing gears (not shown). The distal end of the transmission cable 48 of the transmission assembly 42 is attached to the bell crank 64 at a clevis 68. When the transmission assembly 42 is operated by the handle 24, it results in rotation of the bell crank 64 and consequent rotation of the needle arm 58 between the open and closed positions.

The needle assembly 70 is coupled to a needle mount 83 at an end of the needle arm 58. The needle assembly 70 includes a tubular needle body 74, a needle tip 76, and suture 78 coupled to the needle body. The needle body 74 includes a side opening 80 through which the suture 78 extends, a first end 82 at which the needle assembly is coupled to the needle mount 83, and a second end 84 to which the tip 76 is coupled. The tip 76 defines a tissue-piercing taper. The suture 78 may be formed of any materials commonly available for surgical suture such as nylon, polyolefins, PLA, PGA, stainless steel, nitinol, and others. One suitable needle assembly is described in more detail in U.S. Pat. No. 9,198,562, which patent is hereby incorporated herein by reference in its entirety for all purposes.

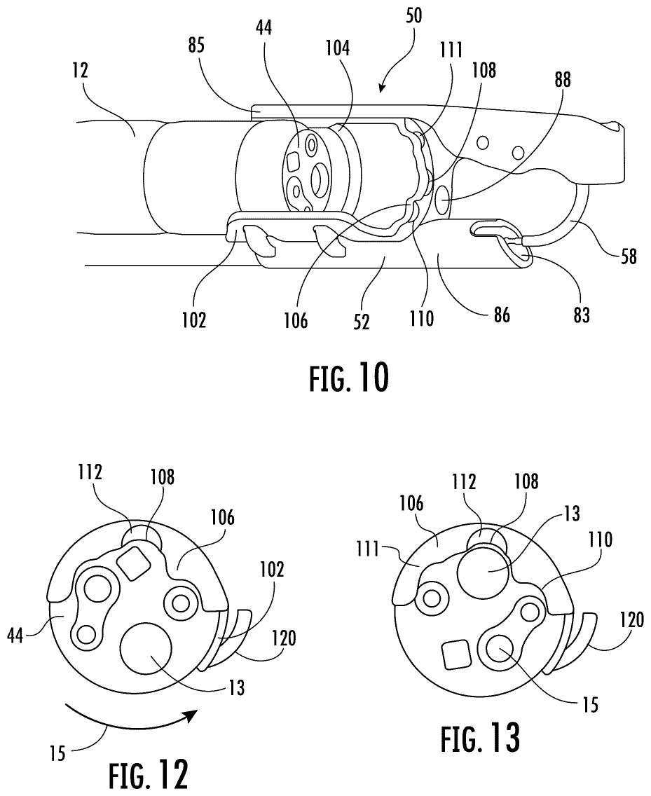

Referring to FIG. 9 and FIG. 10, the mount 52 of the cap assembly 50 includes a side recess 85 into which the transmission assembly 42 is received, and a first throughbore 86 and a second throughbore 88. The first throughbore 86 is positioned in alignment with both the needle mount 83 of the needle arm 58 and needle assembly 70 when the needle arm 58 is in the closed position. A tissue guide 87 extends distally on the mount 52 from over the first throughbore 86 and provides a surface on which to stabilize tissue as it is pierced by the needle assembly 70. The second throughbore 88 is positioned between the first throughbore 86 and the support bracket 54. More particularly, the axial center of the second throughbore 88 is positioned between the first throughbore 86 and the pin 60 (or axis) on which the end effector and/or assembly 58 rotates. The first and second throughbores 86, 88 may be parallel to each other and the longitudinal axis A of the endoscope, or the second throughbore 88 may be obliquely angled relative to the first throughbore 86 so as to direct the second device 40 at a particular orientation into the needle path, as described further below. The mount 52 is structured such that when the cap assembly 50 is coupled to the endoscope 12, as described below, the first and second throughbores 86, 88 are positioned radially outside the profile of the endoscope 12.

Referring to FIG. 8 and FIG. 11, the distal end of the first catheter 34 is fixed in the first throughbore 86, and its proximal end is coupled to a first connector 33 on the handle bracket 26. The distal end of the second catheter 36 is fixed in the second throughbore 88, and its proximal end is coupled the second connector 35 on the handle bracket 26. The first and second catheters 34, 36 may be discrete catheters or may be separate lumens of a common catheter. In a preferred embodiment, the catheters 34, 36 are discrete catheters covered in a preferably crescent moon shaped common sheath 89 along substantially all (e.g., at least 80 percent, and more preferably at least 90 percent) of their length. Alternatively, the common sheath 89 may extend along the entire length of the catheters 34, 36, a partial length thereof, or may be provided in sections along selected portions of the catheters 34, 36. In a preferred embodiment, the common sheath 89 is flexible, defines a lumen 90 for catheter 34, a lumen 92 for catheter 36, and a lumen 94 for both transmission assembly 42 and a retroflex pull string 100, the function of which is described below. Optionally, separate lumens can be provided for the transmission assembly 42 and the pull string 100. A concave recess 96 is provided along the length of the sheath 89 so that the sheath is preferably adapted to extend in close approximation to the exterior of an appropriately sized endoscope 12. In an embodiment, the sheath 89 is adapted to accommodate the endoscope such that the sheath preferably extends more than 180° about a circumference of, and thereby partially surrounds the exterior, of the appropriately sized endoscope 12.

Referring to FIG. 8 and FIG. 9, the first catheter 34 is adapted to receive a first device 38 that has a distal end effector that can receive and grasp the needle assembly 70. The second catheter 36 is adapted to receive a second device 40 that has a distal end effector that can engage tissue, and draw the tissue back into the path of the needle so that the tissue can be pierced by the needle assembly 70 as the needle assembly 70 is moved from the open to the closed position.

Referring to FIG. 10, the cap assembly 50 is secured to the distal end 44 of the endoscope 12 with a peripheral engagement structure that is adapted to be positioned about approximately 180°, and preferably slightly greater than 180°, around the circumference of the distal end of the endoscope 12. In one embodiment, the structure is a resilient clip 102 integrated at the proximal end of the mount 52. The clip 102 includes an opening 104 to allow the distal end 44 of the endoscope 12 access therethrough. The clip 102 may be formed from ABS plastic, other suitable plastics, clastic materials, as well as polymer-coated metals.

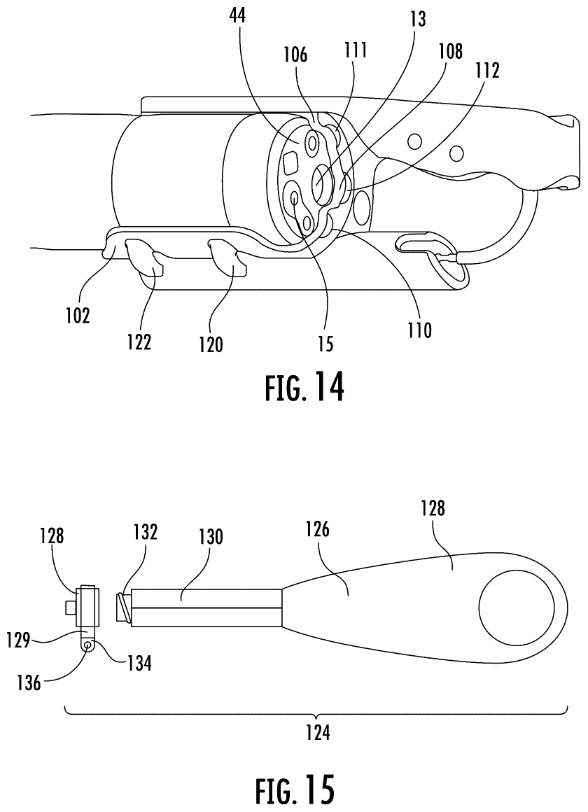

Turning to FIG. 10, FIG. 12, FIG. 13, and FIG. 14, a stop 106 is formed at the distal end of the clip 102. The distal end 44 of the endoscope 12 is adapted to pushed into the recess 104 of the clip 102 and seat against the stop 106. The stop 106 preferably includes a largest first clearance window, formed as a first recess 108 in the circumference of the stop 106 to permit pass through access to instrument channel 13 on various endoscopes of multiple manufacturers when the endoscope is properly rotationally aligned in the clip 102, and smaller second and third clearance windows, formed as second and third recesses 110, 111 approximately 60° on either side of the first recess 108, to prevent obstruction of the optical lens 15 and other working features of various endoscopes. The first recess 108 is located radially inward of the instrument channel lumen 88. In order to align the features of the endoscope relative to such first, second and third recesses 108, 110, 111, the stop 106 includes an indicia 112 relative to which the instrument channel 13 or other designated feature of an endoscope is rotationally aligned. For example, if an endoscope does not include an instrument channel 13 (as such is not required with the system described herein), the optical lens could be designated to be aligned with the indicia 112. In a preferred embodiment, the indicia 112 is a contrasting color relative to a color of a majority of the cap assembly which is provided about the first recess 108. With such rotational alignment of the designated endoscope feature relative to the indicia 112 (in the direction of arrow 115), the optical lens 15 of the endoscope is assured unobstructed view and appropriate orientation relative to the suturing arm 58, instrument channel lumen 88, and needle exchange lumen 86, regardless of the major manufacturer endoscope utilized with the system, without necessitating activating the endoscope. That is, the relative orientation, access, and field of view can be ascertain without requiring powering up the endoscope and previewing the field of view from the lens 13 on the display 14 in advance of the procedure. This significantly reduces preparation time prior to the procedure. The location of the first and second recesses 108, 110 and the indicia 112 relative to the suturing arm 58, instrument channel lumen 88, and needle exchange lumen 86, as well as areas which will be covered and blocked from view at the distal end of the endoscope, is based on analyses of scans of multiple distal ends of endoscopes.

Referring to FIG. 14, the resilient clip 102 further includes a plurality of longitudinally displaced (proximal and distal) integrated hooks 120, 122. In an embodiment, exactly two hooks are provided to the clip. The hooks are preferably identified with contrasting color relative to surrounding areas of the end cap and to a majority of the cap assembly. The hooks 120, 122 may also be numbered with indicia, e.g. “1” and “2”, according to the intended order of use, as further described below.

Turning to FIG. 15, the system also includes a tape applicator 124 for applying tape at each of the hooks 120, 122. The tape applicator 124 includes a handle 126 and a spool 128 of tape 129 on a distal end of the handle. The illustrated example of an embodiment of a handle 126 is preferably monolithic, including a wider proximal end 126p for stable gripping in a user's hand, and a narrower extension 130 for unobtrusively reaching about the distal end 44 of the endoscope 12 and cap assembly 50 during tape application. The extension 130 includes a spool mount 132. In a preferred embodiment the spool mount 132 is in the form of an external thread. The spool 128 is rotatably fixed on the spool mount 132 of the handle 126 during use. The spool 128 includes an internal thread (not shown) that allows the spool to be rotated onto the spool mount 132 in a rotational direction opposite the intended application of the tape, such that when tension is applied to the tape the spool is pulled in a direction that rotationally secures the spool relative to the mount. Other securing interfaces between the mount and spool can be used. The tape preferably has a non-adhesive free end 134 provided with an integrated loop 136 that is reinforced and sized for attachment to one of the hooks 120, 122. The reinforced integrated loop 136 is preferably provided in a color to facilitate locating it in the environment of an operating room. By way of example, the loop 136 is preferably opaque; and the loop preferably matches color with the hooks on the cap assembly. Both may be opaque white. In an embodiment, the loop 136 has a diameter of approximately 2.5 mm. The diameter of the spool 128 is preferably less than the diameter of the endoscope 12 to provide mechanical advantage when wrapping the tape 129 to secure the clip 102 and endoscope 12 relative to each other. In one embodiment, the spool has a diameter of 10 mm and a tape width of 5 mm. In an embodiment, except for the free end 134, the tape 129 has a consistent adhesion along its length and is primarily adapted to stick to the back of itself under tension, as well as both the endoscope 12 and the clip 102. Alternatively, the tape 129 can have a non-stick first portion for first wrapping about the endoscope, and a second adhesive portion for wrapping over and adhering to the back of the first portion and the back of the second portion.

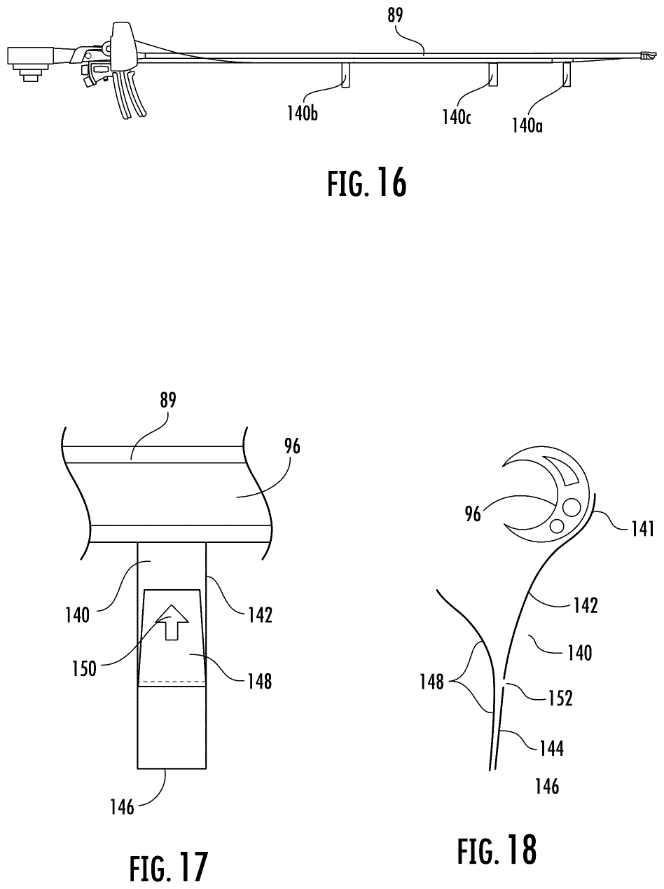

Turning now to FIG. 16, FIG. 17, and FIG. 18, a tape system is also provided for securing a portion of the sheath relative to the endoscope. The sheath 89 is provided with a plurality of strips of tape 140 pre-disposed along the length of the sheath 89. In a preferred embodiment, three strips of tape are used; a first strip 140a located at a distal end of the sheath, a second strip 140b located at a substantially central location along the length of the sheath, and a third strip 140c located between the first and second strips 140a, 140b and preferably closer to the first strip 140a. This limited use of tape and particular spacing has been shown to provide good stabilization of the sheath relative to the endoscope during use. That the, additional strips of tape can be pre-provided to the sheath and used in the manner described. In accordance with one aspect of the system, the strips of tape 140 have an adhered first portion 141 pre-applied to the sheath 89, a second portion 142 close to the sheath recess 96 that is non-sticky to prevent adhesion to the endoscope as the tape is wrapped around the endoscope, and a third portion 144 extending from the second portion 142 and to a free end 146 of the tape strip that is adhesive and adapted to adhere to the back of the tape when it is wrapped around the endoscope 12 and sheath 89. In accordance with another aspect, the tape strips 140 each include a pull off protective strip 148 that covers the second portion 144. The pull off protective strip 148 extends to cover the third portion 144 of the tape and is bent into an L-shaped extending tab between the free end 146 and the first portion 141. The tab 148 is preferably a high visibility color, contrasting with the sheath, e.g., white, and also preferably includes indicia, e.g., a laser-cut arrow 150, indicating the direction in which the tab 148 of the protective strip should be pulled to release the protective strip from the third portion to expose the adhesive (FIG. 17). In accordance with one aspect of the system, the strips 140 have at least a perforation 152 that permits breakaway disassembly after use of the system (FIG. 18). The perforation 152 is preferably provided at or near the junction of the second and third portions 142, 144 of the tape.

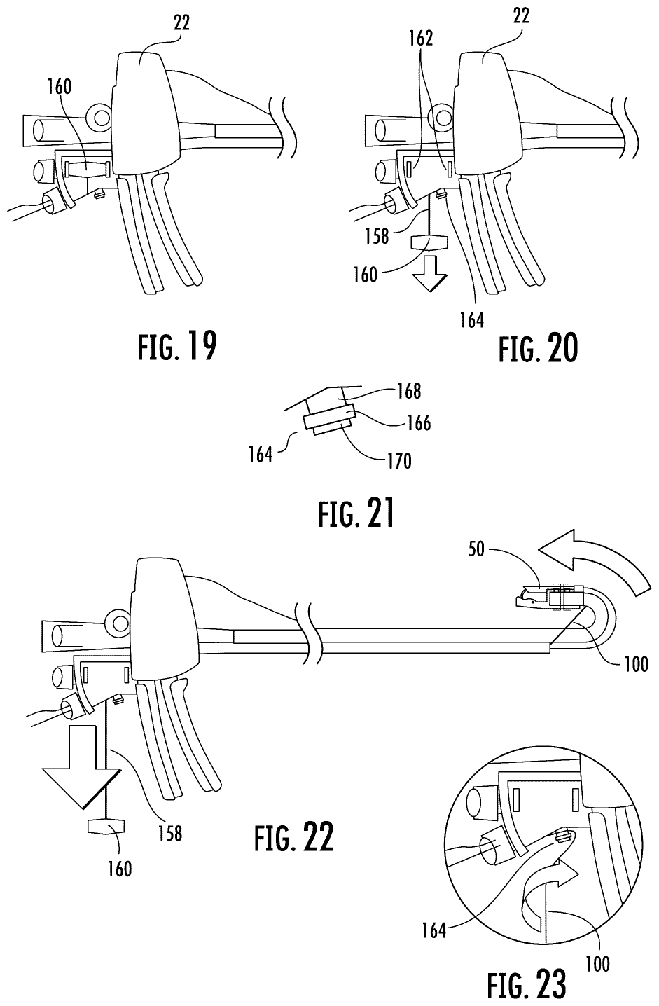

Turning to FIG. 8, FIG. 19, and FIG. 20, in accordance with another aspect of the system, the cap assembly 50 is adapted to retroflex relative to the sheath 89 via actuation from the proximal handle 24. The pull string 100, referenced above, extends from the proximal handle 24, through lumen 94 in the sheath 89, and exits the distal end of the sheath. A pull string 100 herein refers to a textile, polymer, or metal monofilament, multifilament, or cable stranded element. Between the distal end of the sheath and the end cap there is gap 154 (FIG. 8) exposing a length of the catheters 34, 36, the transmission assembly 42, and the pull string 100 outside the sheath 89 and to the cap assembly 50. The length of the gap 154 generally represents the difference in length between the endoscope 12, which has a longer first length, and the sheath 89, which has a shorter second length. In a preferred embodiment the exposed length of the gap 154 is less than 16 cm; more preferably the length of the gap is between 6 and 12 cm; even more preferably the length of the gap is between 7.5 and 10 cm; and most preferably the exposed length of the gap is 8.5 cm to provide optimal retroflexion, as described below. On the opposite side of the gap 154, the transmission assembly 42 is secured in the side recess 85 of the cap assembly 50 and the catheters 34, 36 are secured within the first and second throughbores 86, 88 (e.g., as illustrated in FIG. 9). Further, a distal end 156 of the pull string 100 is secured to the cap assembly 50. In one assembly, the distal end 156 is bonded into a hole (not shown) at the proximal end of the cap assembly 50. The proximal end 158 of the pull string 100 exits from the sheath and through an opening in the proximal handle 24 and is attached to a pull handle 160. A pull handle mount 162 is preferably formed on the proximal handle 24 and can secure the pull handle 160 via interference fit when not in use. A pull string lock 164 is provided on the proximal handle 24 to secure the pull string in a tensioned state. An example of an embodiment of a pull string lock 164 includes a resilient rubber washer 166 secured on a seat 168 and over a post 170 (FIG. 21. In use, when it is desirable to retroflex the cap assembly 50, the pull handle 160 can be released from the handle mount 162 and pulled to apply tension to the pull string 100. As tension is applied to the pull string 100, the pull string 100 is retracted across the gap 154 and through the lumen 94. As the pull string 100 is pulled across the gap 154, the cap assembly 150 is retroflexed to shorten the distance between the end of the sheath 89 and the location at which the distal end 156 of the string is attached to the cap assembly 50, as shown in FIG. 22. When a desired degree of retroflex is obtained, the tension on the pull string 100 may be temporarily maintained (and the retroflex configuration fixed) by wrapping the proximal end 158 of the pull string 100 around the post 170 between the washer 166 and the seat 168 (FIG. 23). Compression of the pull string 100 between the washer 166 and the seat 168 retains the pull string in position. The pull handle 160 can be placed back on the handle mount 162 to keep it out of the way (i.e., back to the position shown in FIG. 19) while other features of the proximal handle 24 and the endoscope 12 are used. Retroflex of the system may be used in combination with actuating retroflex of the endoscope. Then, when it is intended to release the retroflex condition at the distal end, the proximal end 158 of the pull string 100 is unwound from the pull string lock 164, and tension on the pull string is released. In such manner, retroflex of the system operates as an assist to the system attached to the endoscope and ensures that retroflex of the endoscope is carried out full in the manner intended. It is also recognized that this type of retroflex system, with or without assist from the endoscope, can be used in different endoscopic therapeutic and interventional systems, and is not limited to systems for suturing.

In light of the above, the suturing device may be prepared for use in conjunction with an endoscope as follows. The distal end 44 of the endoscope 12 is pushed into the resilient clip 104 until seated at or near the distal stop 106 (FIG. 10). Then, the endoscope and cap assembly 50 are coaxially rotated relative to each other until the instrument channel 13 of the endoscope is rotationally aligned with the indicia 112 at the distal stop 106 to ensure appropriate alignment between the two (FIG. 12 and FIG. 13). Then, if necessary, the endoscope 12 is pushed all the way into contact with the distal stop 106 (FIG. 14).

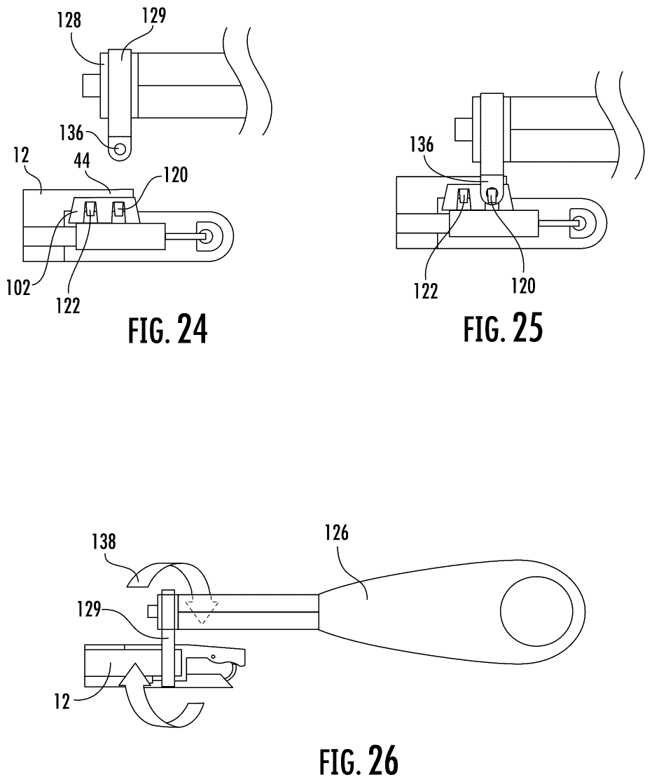

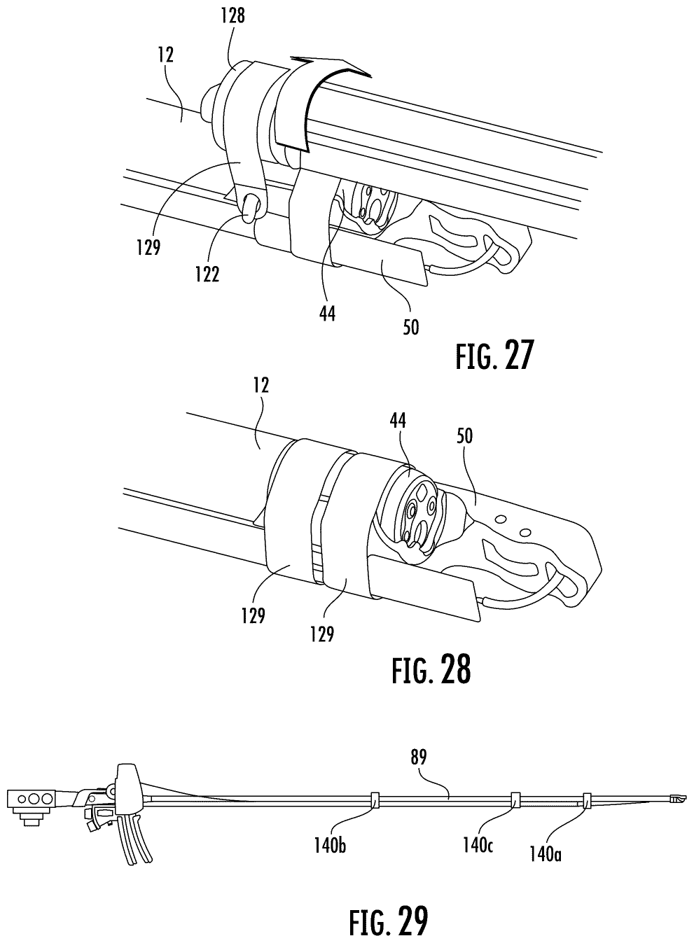

Then, referring to FIG. 24, FIG. 25, and FIG. 26, the loop 136 at the free end 134 of a first spool 128 of a tape applicator 124 is engaged over the distal first hook 120 and the handle 126 coupled to the spool 128 is maneuvered to wrap the tape 129 over a distalmost portion of the distal end 44 of the endoscope 12, over the clip 102, and back and around over the back of the tape 129 in a circular motion (arrow 138) one or more times to secure the endoscope relative to the clip. While wrapping, tension is applied to the tape. The relative diameters of the tape spool 128 and the endoscope 12 facilitate application of significant tension on the tape during wrapping of the endoscope with the tape. After the tape is applied at the first hook 120, the depleted spool 128 is removed from the spool mount 132, and a fresh spool 128 of tape is attached at the spool mount of the handle 126. Referring to FIG. 27 and FIG. 28, the tape 129 from the fresh spool 128 is similarly applied starting from the second hook 122 and over an adjacent area of the endoscope 12 until the distal end 44 of the endoscope 12 and cap assembly 50 are secured together.

Then, turning to FIG. 11, FIG. 17, FIG. 18, and FIG. 29, the endoscope is fully pressed into the recess 96 along the length of the sheath 89. Starting from the distal end of the system, and at strip of tape 140a, the tab 148 of the protective strip is pulled off to expose the adhesive portion 144, the tape 140 is pulled taut, the first portion 142 of the tape is wrapped around the endoscope 12, and the second adhesive portion 144 of the tape is adhered to the sheath on the opposite side of the recess 96 and then extended over the tape again to secure the endoscope thereat. The process is repeated along the length of the sheath for remaining strips 140c, then 140b, to properly secure the sheath relative to the endoscope. This longitudinally secures the sheath 89 to the exterior of the endoscope 12 for in vivo operation.

The collar 28 is properly positioned at the proximal handle 45 of the endoscope 12. The first device, a needle capture instrument 38 loaded with a needle assembly 70, is advanced through the first port 32, into the first lumen 34 and to the cap assembly 50. Suitable needle capture devices 38 are described in detail in U.S. Pat. No. 8,679,136, which patent is hereby incorporated herein by reference in its entirety for all purposes. The needle assembly 70 is loaded onto the needle arm 58, with the suture 78 extending parallel to the needle capture instrument 38 within the first lumen 34.

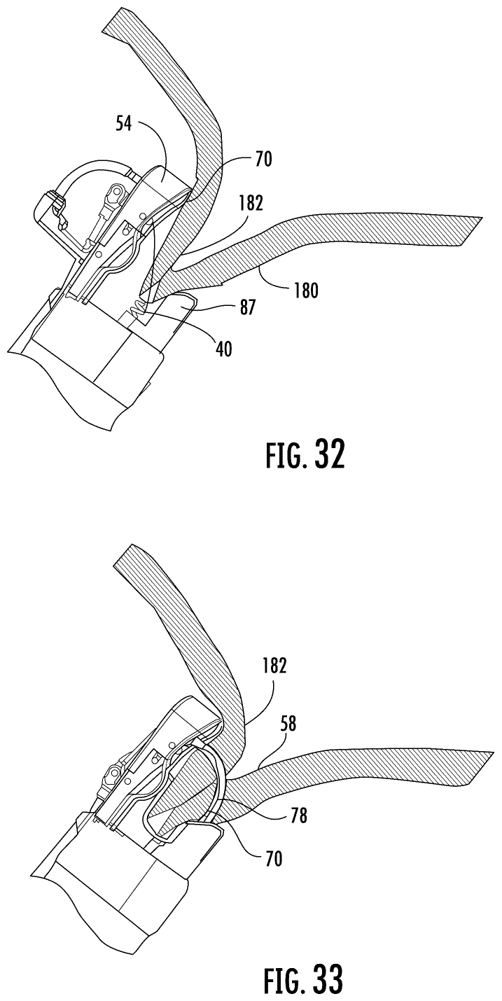

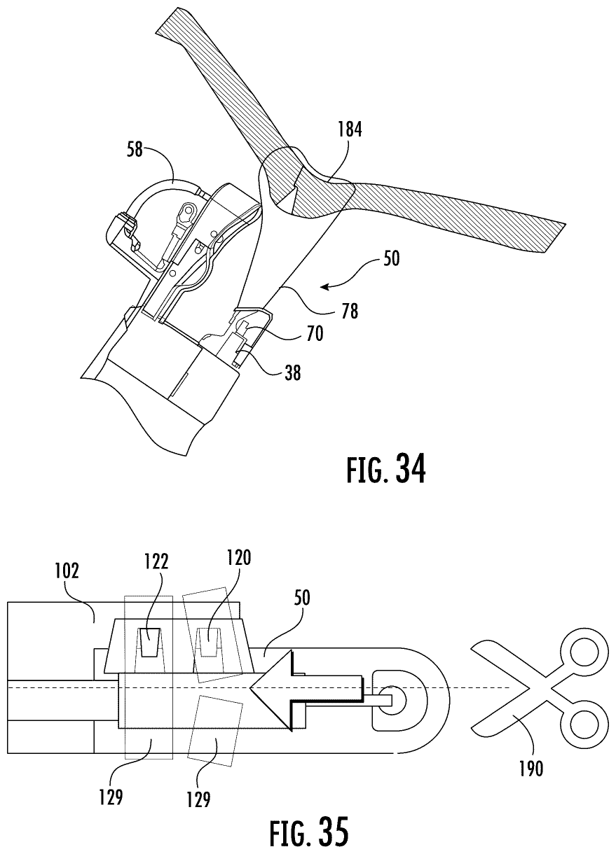

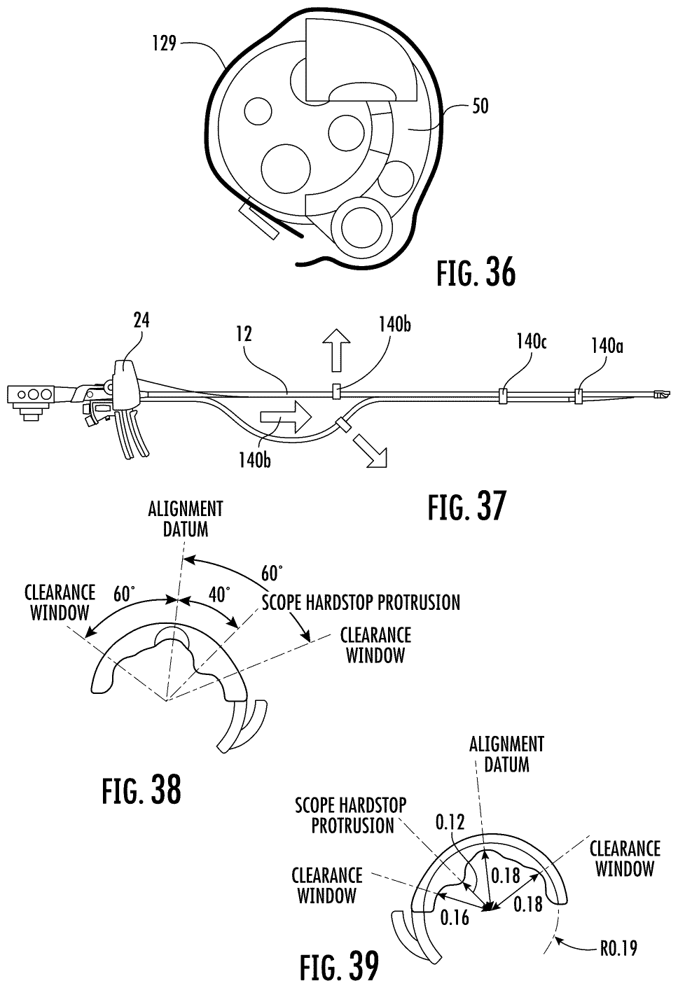

With reference to FIGS. 1 and 25, the distal end of the endoscope 12 and cap assembly 50 of the suturing device 22 are advanced into a natural orifice of a patient, optionally through a guide tube (not shown), and approached to target tissue 180. If at any point it is necessary to reorient the endoscope 12 via retroflexion, the cap assembly 50 can be retroflexed as well, as described above, to aid and/or further direct the distal end of the endoscope and the cap assembly to the intended target tissue. Once adjacent the target tissue 180, the handle 24 of the suturing device 22 is operated to move the needle arm 58 into the open position, as shown in FIG. 29. Turning to FIG. 31, the end effector of the second device, e.g., a tissue retractor 40 having a helical coil 40a at its distal end, is advanced through the second port 32, into the second catheter 36 (FIG. 8) and out the second throughbore 88 (FIG. 9), and beyond the cap assembly 50. Suitable tissue retractor instruments are described in detail in U.S. Pat. No. 11,083,364, which patent is hereby incorporated herein by reference in its entirety for all purposes. Other tissue retractors, including forceps, may also be used. The helical coil 40a is operated to engage target tissue 180. The tissue retractor 40 is withdrawn to draw the tissue 180 against the tissue guard 87 and into a tissue fold 182 located within the path of the needle assembly 70; i.e., between the bracket 54 and needle guide 87, as shown in FIG. 32. The orientation of the second throughbore 88, either parallel or obliquely angled relatively to the first througbore 86, is adapted to guide the tissue retractor to engage and retract tissue into the needle path. The handle 24 is then operated to move the needle arm 58 into the closed position, thereby piercing the tissue fold 182 and passing the needle assembly 70 with suture 78 through the tissue fold during the movement (FIG. 33). When the needle arm 58 is in the closed position, the needle is received within the distal end of the needle capture device 38 (FIG. 9). The needle capture device 38 is operated to securely engage the needle 70. The handle 24 is then operated to move the needle arm 58 toward the open position, thereby disengaging the needle arm 58 from the needle assembly 70, which remains in the needle capture device 38 (FIG. 34). The tissue retractor 40 is also released from the tissue and withdrawn back through the second catheter 36. The endoscope 12 is then moved to displace the cap assembly 50 relative to the sutured tissue 184. The needle 70 and suture 78 may be secured onto the tissue, such as by knotting or cinching, or the needle may be repositioned on the needle arm and additional suture loops may be formed within adjacent or other areas of tissue. Once the suturing is complete, the needle arm 58 is returned to a closed position, and the endoscope 12 and suturing device 22 are removed from the patient.

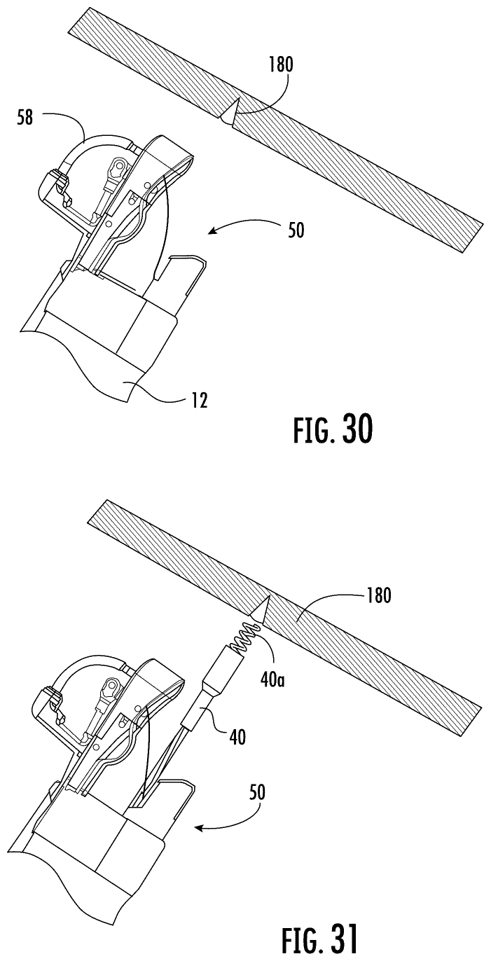

The suturing assembly is then released from over the endoscope by cutting, e.g., with a scissors 190, the two bands of tape 129 coupled to the hooks 120, 122 that secure the endoscope 12 relative to the cap assembly 50, and then unwrapping the tape 129 over the opening of the cap clip 102, as shown by both FIGS. 30 and 31. Then, referring to FIG. 37, the tape strips 140b, 140c, 140a are broken apart, in turn, by applying force in the direction of arrow 174 to break each respective piece of tape at its perforation 152 (FIG. 18); as each tape strip was not directly adhered to the endoscope but only to itself and the sheath 89, the endoscope can be released once the strips are broken. The proximal handle 24 is then fully released from the endoscope 12.

In view of the above, in accordance with various principles of the present disclosure, an example of an embodiment of an endoscopic apparatus for use with an endoscope has a proximal handle assembly; a distal cap assembly; and a central portion adapted to extend along the endoscope between the handle assembly and the cap assembly. In some aspects, the handle assembly defines a recess in which the distal end of the endoscope is received. In some embodiments, the handle includes at least one hook at one side of the recess for receiving a tensile member that wraps around a portion of the cap assembly and the endoscope.

In some embodiments, the at least one hook is identified with a contrasting color relative to a majority of the cap assembly.

Additionally or alternatively, in some embodiments, the distal cap assembly includes a distal stop against which the distal end of the endoscope is intended to be abutted. In some embodiments, the distal stop includes indicia relative to which a feature of the endoscope is intended to be rotationally aligned. In some embodiments, the indicia include a contrasting color relative to a majority of the cap assembly. In some embodiments the indicia on the distal stop and the at least one hook are identified with a common color. In some embodiments, the feature of the endoscope with which the indicia are to be aligned is an instrument channel of the endoscope.

Additionally or alternatively, in some embodiments, the cap assembly is a suturing assembly including a movably mounted needle holder. In some aspects, operation of the proximal handle assembly moves the needle holder on the cap assembly.

In accordance with various principles of the present disclosure, an example of an embodiment of an endoscopic apparatus for use with an endoscope includes a proximal handle assembly; a distal cap assembly; and a central portion adapted to extend along the endoscope between the handle assembly and the cap assembly. In some aspects, the cap assembly defines a recess in which the distal end of the endoscope is received. Additionally or alternatively, the cap assembly includes two longitudinally spaced apart hooks at one side of the recess.

Additionally or alternatively, in some embodiments, each of the hooks are identified by a contrasting color relative to a color of a majority of the cap assembly.

Additionally or alternatively, in some embodiments, each of the hooks are identified by different hook indicia. In some embodiments, the hook indicia indicate an order of operation in which the hooks are intended to be used. In some embodiments, the hook indicia include numbers.

Additionally or alternatively, in some embodiments, the distal cap assembly includes a resilient clip that defines the recess.

Additionally or alternatively, in some embodiments the distal cap assembly includes a distal stop against which the distal end of the endoscope is intended to be abutted, the stop including contrasting alignment indicia relative to which a feature of the endoscope is intended to be rotationally aligned.

Additionally or alternatively, in some embodiments the distal cap assembly includes a distal stop against which the distal end of the endoscope is intended to be abutted, the stop including contrasting alignment indicia relative to which an instrument channel of the endoscope is intended to be rotationally aligned. In some embodiments the distal stop includes first and second recesses, and the alignment indicia is provided at the first recess.

In some embodiments, the cap assembly is a suturing assembly including a movably mounted needle holder, and operation of the proximal handle assembly moves the needle holder on the cap assembly.

In accordance with various principles of the present disclosure, an example of an embodiment of a securing system for use with an endoscope includes an endoscopic apparatus including a proximal handle assembly, a distal cap assembly; a central portion adapted to extend along the endoscope between the handle assembly and the cap assembly; and a spool of tape. In some embodiments, the distal cap assembly defines a recess in which the distal end of the endoscope is received and a hook at a side of the recess. In some embodiments, the spool of tape has a free end provided with a preformed loop adapted to be received over the hook. In some embodiments, the loop is adapted to be attached over the hook. In some embodiments, the tape is adapted to be wrapped about a portion of the cap assembly and the endoscope to couple the cap assembly and endoscope relative to each other.

In some embodiments, the system further includes a tape applicator including a handle and an extension with a spool mount. In some embodiments, the spool is coupled to the spool mount. In some embodiments, the spool is removably coupled to the spool mount.

In some embodiments, the cap assembly includes at least two longitudinally displaced hooks. In some embodiments, the securing system includes at least two spools of tape.

In some embodiments, the hook is identified by a contrasting color from a color of a majority of the cap assembly.

In some embodiments, the cap assembly includes at least two hooks. In some embodiments, each of the hooks is identified by different hook indicia. In some embodiments, the hook indicia identify an order of operation in which the hooks are intended to be used. In some embodiments, the hook indicia include a contrasting color from a majority of the cap assembly.

In some embodiments, the cap assembly is a suturing assembly including a movably mounted needle holder. In some embodiments, operation of the proximal handle assembly moves the needle holder on the cap assembly.

In accordance with various principles of the present disclosure, a method includes coupling a cap assembly of an endoscopic apparatus to an endoscope having a distal end. In some aspects, the cap assembly defines a recess in which the distal end of the endoscope is received. Additionally or alternatively, the cap assembly has first and second longitudinally displaced hooks at one side of the recess. In accordance with various principles of the present disclosure, the method includes inserting the distal end of the endoscope into the recess; providing a first length of tape having a first loop at a free end; coupling the first loop to the first hook; and wrapping at least a portion of the first length of tape about the cap assembly and the distal end of the endoscope.

In some aspects, the method further includes providing a second length of tape having a second loop at a free end; coupling the second loop to the second hook; and wrapping at least a portion of the second length of tape about the cap assembly and the distal end of the endoscope. In some aspects, the first hook is distally displaced from the second hook. In some aspects, the endoscopic apparatus includes a proximal handle and a central longitudinal sheath portion extending between the handle and the cap assembly. In some aspects, the central longitudinal portion includes a concave recess along its length. In some aspects, the method further includes inserting the endoscope into the recess of the central longitudinal sheath portion; and securing the central longitudinal sheath portion to the endoscope. In some aspects, the sheath portion is secured to the endoscope with a plurality of longitudinally spaced apart pieces of tape positioned at least partially circumferentially about the sheath portion and the endoscope. In some aspects, the central longitudinal sheath portion is crescent shaped and defines at least one lumen. In some aspects, at least one lumen includes a first lumen through which a flexible first catheter extends to the cap assembly, and a second lumen through which a flexible second catheter extends to the cap assembly. In some aspects, the cap assembly includes an actuation arm movably mounted thereon. In some aspects, the central longitudinal sheath portion includes a third lumen through which an actuation assembly extends. In some aspects, the actuation assembly is coupled between the proximal handle and the actuation arm and adapted for moving the actuation arm on the cap assembly upon operation of the proximal handle. In some aspects, the endoscopic apparatus includes a pull string having a proximal end and a distal end, the pull string extending from the proximal handle, through the central longitudinal sheath portion, and fixed to the cap assembly. In some aspects, the proximal end of the pull string is retracted relative to the proximal handle, and the distal end of the pull string is tensioned to cause the first and second catheters to flex and draw the cap assembly into retroflexion.

In some aspects, the cap assembly is a suturing assembly including a movably mounted needle holder.

In accordance with various principles of the present disclosure, an example of an embodiment of an endoscopic apparatus for use with an endoscope includes a proximal handle assembly; a distal cap assembly; a sheath portion; and a securing system. In some aspects, the cap assembly defines an assembly for positioning adjacent the distal end of the endoscope. In some aspects, the sheath portion defines a longitudinal recess. In some aspects, the sheath portion is adapted to extend along a portion of an exterior of the endoscope between the handle assembly and the cap assembly. In some aspects, the sheath portion is adapted to extend at least partially about the endoscope at the recess. In some aspects, the recess has a first side and a second side. In some aspects, the securing system is configured to secure the central portion to the exterior of the endoscope during in vivo operation of the endoscope. In some aspects, the securing system includes a plurality of longitudinally spaced apart lengths of tape pre-applied to the sheath portion on the first side of the recess. In some aspects, at least some of or each of the lengths of tape has an adhesive first portion at which the tape is adhered to the sheath on the first side of the recess, and a non-adhesive second portion extending from the first portion. In some aspects, at least some of or each of the lengths of tape has an adhesive third portion extending from the second portion and terminating in a free end.

In some aspects, the securing system further includes a removable non-adhesive protective strip over the third portion.

In some aspects, the protective strip extends into a tab with a free end protruding between the free end and the first portion.

Additionally or alternatively, the protective strip extends into an L-shaped non-adhesive tab. Additionally or alternatively, the tab has indicia indicating a direction in which the tab should be pulled to expose the third portion.

Additionally or alternatively, the protective strip has a contrasting color to the sheath portion.

In some aspects, each length of tape has a perforation between the, at, or near a junction of the second and third portions of the tape.

In some aspects, the securing system includes a first length of tape pre-applied at a distal end of the sheath portion, a second length of tape pre-applied at a central portion of the sheath portion, and a third length of tape pre-applied to the sheath portion between the first and second lengths of tape.

In some aspects, the sheath portion defines at least one lumen in a wall of the sheath portion.

In some aspects, the cap assembly is a suturing assembly including a movably mounted needle holder. In some aspects, operation of the proximal handle assembly moves the needle holder on the cap assembly.

In accordance with various principles of the present disclosure, a method of securing an endoscopic apparatus to an endoscope includes securing an endoscope having an external sheath to an endoscope. In some aspects, the endoscope has a circumference and a first length, and the sheath has a second length configured to extend about at least a portion of the circumference of the endoscope along at least a portion of the first length of the endoscope. In some aspects, the method includes providing the sheath with pre-applied portions of tape along second length; extending the sheath about the portion of the circumference of the endoscope along the first length; and wrapping the pre-applied portions of tape about a periphery of the endoscope and sheath to secure the sheath to the endoscope.

In some aspects, at least one or each pre-applied portion of tape includes an adhesive first portion at which the portion of tape is adhered to the sheath. Additionally or alternatively, at least one or each pre-applied portion of tape includes a non-adhesive second portion extending from the first portion. Additionally or alternatively, at least one or each pre-applied portion of tape includes an adhesive third portion extending from the second portion and terminating in a free end. Additionally or alternatively, at least one or each pre-applied portion of tape includes a removable protective strip over the third portion. In some aspects, the method further includes removing the removable protective strip from other the third portion, extending the second portion into contact with the endoscope, and adhering the third portion into contact with the sheath on an opposite side endoscope relative to the first portion.

In some aspects, the sheath defines a recess, the endoscope is inserted into the recess, and the first and third portions of the tape are located on opposite sides of the recess.

In some aspects, each pre-applied portion of tape includes a perforation. In some aspects, the method further includes releasing the endoscope from the sheath by applying a force to break the tape at the perforation. In some aspects, the tape is broken without cutting. In some aspects, the tape is broken by applying a force parallel to a longitudinal axis of the endoscope. In some aspects, the cap assembly is a suturing assembly including a movably mounted needle holder.

In accordance with various principles of the present disclosure, an example of an embodiment of an endoscopic apparatus for use with an endoscope includes a proximal handle assembly; a distal cap assembly; a sheath; a first catheter; a second catheter; and a transmission cable. In some aspects, the distal cap assembly is adapted to be positioned adjacent the distal end of the endoscope. In some aspects, the cap assembly has an end effector movable thereon. In some aspects, the sheath is a crescent-shaped flexible sheath. Additionally or alternatively, the sheath has a longitudinal side-opening recess sized to receive a portion of the endoscope between the handle assembly and the cap assembly. Additionally or alternatively, the sheath defines a first lumen, a second lumen, and a third lumen. In some aspects, the first catheter extends in and distally beyond the first lumen to the cap assembly. In some aspects, the second catheter extends in and distally beyond the second lumen to the cap assembly. In some aspects, the transmission cable extends from the handle assembly. In some aspects, the transmission assembly extends from the handle assembly into the third lumen. In some aspects, the transmission assembly extends distally beyond the third lumen to the cap assembly. In some aspects, the transmission assembly is operably coupled to the end effector. In some aspects, operation of the handle assembly moves the transmission cable to operate the end effector.

In some aspects, the cap assembly is a suturing assembly and the end effector is a needle holder.

In accordance with various principles of the present disclosure, an example of an embodiment of an endoscopic apparatus is configured for use with an endoscope having a proximal end and a distal end, an instrument channel, and a lens. In accordance with various principles of the present disclosure, the endoscopic apparatus includes a proximal handle; a distal cap assembly; and a central portion extending between the handle and cap assembly. In some aspects, the distal cap assembly defines an assembly for interacting with tissue. In some aspects, the distal cap assembly is adapted to be positioned adjacent the distal end of the endoscope. In some aspects, the distal cap assembly includes a clip to receive the distal end of the endoscope. In some aspects, the distal cap assembly includes a stop to limit distal travel of the endoscope. In some aspects, the stop has indicia against which a feature of the endoscope is rotationally aligned to ensure appropriate orientation of the lens and instrument channel of the endoscope without obstruction thereof. In some aspects, the central portion extends between the handle and cap assembly.

In some aspects, the stop is adapted to function with endoscopes from multiple manufacturers.

In some aspects, the cap assembly is a suturing assembly including a movably mounted needle holder. In some aspects, operation of the proximal handle moves the needle holder on the cap assembly.

In accordance with various principles of the present disclosure, an example of an embodiment of an endoscopic apparatus for use with an endoscope includes a proximal handle assembly; a distal cap assembly; a sheath; at least one catheter; and a pull string. In some aspects, the distal cap assembly is adapted to couple to the distal end of the endoscope spaced from the handle assembly by a first length. In some aspects, the sheath is a flexible sheath. In some aspects, the sheath has a proximal end and a distal end, the proximal end attached to the handle assembly. In some aspects, the sheath extends a second length less than the first length. In some aspects, the difference between the first length and the second length defines a gap. In some aspects, the sheath is adapted to extend at least partially about an exterior of the endoscope. In some aspects, the sheath defines at least one lumen. In some aspects, the at least one catheter extends through the at least one lumen from the proximal end of the sheath. In some aspects, the at least one catheter extends beyond the distal end of the sheath. In some aspects, the at least one catheter is fixed to the cap assembly. In some aspects, the pull string has a proximal end and a distal end. In some aspects, the pull string extends from the proximal end of the sheath, through the at least one lumen, out of the distal end of the sheath. In some aspects, the distal end of the pull string is fixed to the cap assembly. In some aspects, the proximal end of the pull string extends from an opening in the handle assembly. In some aspects, when the proximal end of the pull string is retracted relative to the handle, the distal end of the pull string is tensioned to cause the catheter to flex and draw the cap assembly into retroflexion across the gap.

In some aspects, the distal cap assembly is a suturing assembly including a needle movable relative to the at least one catheter. In some aspects, the pull string extends through a common lumen with a transmission cable for operating the suturing assembly.

In some aspects, the apparatus includes a pull handle attached to the proximal end of the pull string. In some aspects, the proximal handle assembly is adapted to releasably store the pull handle.

In some aspects, the apparatus further includes a locking system to temporarily retain the pull string under tension. In some aspects, the proximal handle assembly includes the locking system. In some aspects, the locking system includes a post having a seat and a washer provided over the post on the seat. In some aspects, the proximal end of the pull string is retained in position on the post. In some aspects, the proximal end of the pull string is retained in position on the post when wrapped around the post between the seat and the washer. In some aspects, the washer is made of a resilient material.

In accordance with various principles of the present disclosure, an example of an embodiment of an endoscopic system includes an endoscope and an external instrument channel device couplable to the endoscope. In some aspects, the endoscope has a proximal end and a distal end and an outer surface extending between the proximal and distal ends. In some aspects, the proximal end has a first handle. In some aspects, the distal end is adapted to retroflex upon actuation of the proximal end. In some aspects, the instrument channel device has a second handle. In some aspects, the instrument channel device has a sheath adapted to be coupled in close proximity to the outer surface of the endoscope between the proximal and distal ends. In some aspects, the instrument channel device has a cap removably coupled to the distal end of the endoscope. In some aspects, the instrument channel device has an instrument channel extending from the distal end of the sheath to the cap. In some aspects, the instrument channel device has a pull string extending from the second handle, through the sheath, and fixed relative to the cap. In some aspects, when the pull string is placed under tension, the cap is biased into a retroflex position about the instrument channel.

In some aspects, the cap is part of a suturing assembly.

In some aspects, the system further includes a pull handle attached to the proximal end of the pull string. In some aspects, the external instrument channel device includes a proximal handle assembly. In some aspects, the proximal handle assembly is adapted to releasably store the pull handle.

In some aspects, the system further includes a locking system to temporarily retain the pull string under tension. In some aspects, the external instrument channel device includes a proximal handle assembly, and the proximal handle assembly is provided with the locking system. In some aspects, the locking system includes a post having a seat and a washer provided over the post on the seat. In some aspects, the proximal end of the pull string is retained in position on the post. In some aspects, the proximal end of the pull string is retained in position on the post when wrapped around the post between the seat and the washer.

The suturing assemblies described above are adapted for use with an endoscope that does not necessarily have at least two instrument channels. As such, the suturing system can be used with smaller endoscopes that are available in many surgical settings and which can be more easily advanced through a natural orifice. In addition, as indicated aspects of the system can be used in other surgical treatment settings other than for suturing.

There have been described and illustrated herein embodiments of a suturing system as well as a surgical treatment system, as well as methods of using the same. While particular embodiments of the invention have been described, it is not intended that the invention be limited thereto, as it is intended that the invention be as broad in scope as the art will allow and that the specification be read likewise. Thus, while particular instruments and devices for advancement through the first and second lumen have been disclosed, it will be appreciated that other instruments can also be used through such lumen for like or even different purpose. Also, while the treatment system has been particularly described with respect to a cap assembly having an end effector in the form of a needle arm that carries a needle, it is recognized that alternatively one or more movable end effectors with other structure and purpose can be provided to the cap assembly. Also, while a tissue anchor in the form of a needle assembly has been described, the end effector can deploy different types of tissue anchors, including, e.g., clips. In addition, while a particular needle assembly has been described, other needle assemblies can similarly be used. Also, the size and instrument channel features of the endoscope with which the system is used is not critical, it is appreciated that various prior art systems cannot be properly used in a suturing operation in conjunction with endoscopes having fewer than two instrument channels, one for receiving a needle exchange device and the other for receiving a tissue retractor, whereas the present system is capable of complete operation without the provision of any channels through the endoscope. Further, while it is indicated that various features described herein are not limited to suturing applications, such as the retroflex system, it is specifically recognized that the retroflex system may be used in association with a cap assembly adapted to provide various other surgical applications including, but not limited to, staplers, clip appliers, band ligators, tissue manipulating instruments, cutting instruments, forceps, biopsy instruments, injection devices, as well as cap assemblies that have no significant function other than to support external catheters for passthrough of instruments. It will therefore be appreciated by those skilled in the art that yet other modifications could be made to the provided invention without deviating from its scope as claimed.

It is to be understood by one of ordinary skill in the art that the present discussion is a description of illustrative examples of embodiments only, and is not intended as limiting the broader aspects of the present disclosure.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims. Various further benefits of the various aspects, features, components, and structures of devices and systems such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A flexible tubular elongate member for use with a retroflexion system, said flexible tubular elongate member comprising:
an exterior surface and an interior surface extending between a proximal end and a distal end of said flexible tubular elongate member, the interior surface defining one or more lumens through said flexible tubular elongate member between the proximal end and the distal end of said flexible tubular elongate member; and
a flexion section along the length of said flexible tubular elongate member between the proximal end and the distal end configured to be kink-resistant and/or deformation resistant;
wherein:
at least one of the exterior surface or the interior surface of said flexion section is shaped, configured, and/or dimensioned to facilitate flexing of said flexion section.

2. The flexible tubular elongate member of claim 1, wherein at least one of the exterior surface or the interior surface of said flexion section is corrugated.

3. The flexible tubular elongate member of claim 2, wherein said flexion section is reinforced.

4. The flexible tubular elongate member of claim 3, wherein said flexion section includes a wire coil between the exterior surface and the interior surface thereof reinforcing said flexion section.

5. The flexible tubular elongate member of claim 4, wherein the exterior surface and the interior surface are corrugated as a result of the shape of said wire coil therebetween.

6. The flexible tubular elongate member of claim 1, wherein said flexion section includes a wire coil between the exterior surface and the interior surface thereof to facilitate flexion of said flexion section.

7. The flexible tubular elongate member of claim 6, wherein said flexion section further comprises an outer tubular core layer over said wire coil\ and an inner tubular core layer within said wire core, thereby forming a multi-layer wall of said flexion section.

8. The flexible tubular elongate member of claim 1, wherein one or more wall regions of said flexion section has alternating thick and thin wall thicknesses.

9. The flexible tubular elongate member of claim 1, wherein the cross-sectional shape of the one or more lumens through said flexion section remains substantially the same as said flexion section is flexed.

10. The flexible tubular elongate member of claim 1, wherein said flexion section extends along a portion of a distal end of said flexible tubular elongate member.

11. A system for retroflexing a flexible elongate member of a medical device, said system comprising:
an elongated actuation component extending along said flexible elongate member and having a proximal end and a distal end;
a distal mount configured to be mounted with respect to the distal end of the flexible elongate member;
a pivot mount configured to be mounted with respect to the flexible elongate member proximal to the distal mount; and
an auxiliary flexible tubular elongate member configured to extend along the flexible elongate member and having a proximal end and a distal end;
wherein:
the distal end of said auxiliary flexible tubular elongate member is operatively associated with said distal mount;
the distal end of said actuation component is operatively associated with said distal mount to cause flexion of the distal end of the flexible elongate member when said actuation component is pulled proximally;
an actuation region of said actuation component pivots with respect to said pivot mount when said actuation component is pulled proximally to cause the flexible elongate member to retroflex;
said auxiliary flexible tubular elongate member comprises a flexion section operatively associated with said distal mount to flex when said actuation component is pulled proximally;
said flexion section is configured to be kink-resistant and/or deformation resistant when flexed; and
one or more lumens are defined through said flexion section and have a cross-sectional shape unaffected and unchanged by flexing of said flexion section.

12. The system of claim 11, further comprising a flexible tubular elongate member having a proximal end and a distal end with a lumen extending therebetween:
wherein:
said actuation component extends through the flexible tubular elongate member lumen;
said flexible tubular elongate member is configured to be mounted with respect to the flexible elongate member with the distal end of the flexible tubular elongate member proximal to the distal end of the flexible elongate member;
the distal end of said flexible tubular elongate member forms said pivot mount for said actuation component; and
said auxiliary flexible tubular elongate member extends along said flexible tubular elongate member with said flexion section extending distally beyond the distal end of the distal end of said flexible tubular elongate member to be operatively associated with said distal mount.

13. The system of claim 12, wherein said flexion section of said flexible tubular elongate member does not extend to the proximal end of said flexible tubular elongate member.

14. The system of claim 12, wherein said flexible tubular elongate member extends through a lumen defined through said flexible tubular elongate member.

15. The system of claim 11, wherein:
said flexion section has an exterior surface and an interior surface, the interior surface defining the lumen through said flexion section; and
at least one of the exterior surface or the interior surface of said flexion section is shaped, configured, and/or dimensioned to facilitate flexing of said flexion section.

16. The system of claim 11, wherein said flexion section is reinforced with a wire coil.

17. A method of forming a kink-resistant and/or deformation-resistant flexion section of a flexible tubular elongate member insertable transluminally into a patient, said method comprising corrugating at least one of an exterior surface or an interior surface of the flexion section to increase flexibility of the flexion section so that a cross-sectional shape of one or more lumens defined through the flexion section is not changed or affected by flexion of the flexion section.

18. The method of claim 17, further comprising corrugating both the exterior surface and the interior surface of the flexion section.

19. The method of claim 18, further comprising reinforcing the flexion section with a wire coil.

20. The method of claim 19, further comprising forming the flexion section with an outer tubular core layer over the wire coil to form the exterior corrugated surface of the flexion section, and providing the wire coil over an inner tubular core layer to form the interior corrugated surface of the flexion section.
